(19) United States
(12) Patent Application Publication
(10) Pub. No.: US 2025/0101077 A1
(43) Pub. Date: Mar. 27, 2025

(54) PEPTIDES AND PHARMACEUTICAL COMPOSITION FOR THE TREATMENT OF TUMOURS

(71) Applicant: UNIVERSIDAD DE SALAMANCA, Salamanca (ES)

(72) Inventors: Aránzazu TABERNERO URBIETA, Salamanca (ES); Claudia Casilda OLLAURI IBÁÑEZ, Salamanca (ES); Andrea ÁLVAREZ VÁZQUEZ, Salamanca (ES); Sara GUTIÉRREZ PELAZ, Salamanca (ES); Myriam JARAÍZ RODRÍGUEZ, Salamanca (ES); Raquel FLORES HERNÁNDEZ, Salamanca (ES); María PANIAGUA SANCHO, Salamanca (ES)

(21) Appl. No.: 18/729,981

(22) PCT Filed: Jan. 19, 2023
(86) PCT No.: PCT/EP2023/051210

§ 371 Date: Jul. 18, 2024

(30) Foreign Application Priority Data
Jan. 19, 2022 (ES) P202230039

(51) Int. Cl. C07K 14/705; A61K 38/00; A61P 35/00

(52) U.S. Cl. CPC C07K 14/705; A61P 35/00; A61K 38/00; C07K 2319/10

(57) ABSTRACT

The present invention relates to peptides comprising the amino acid sequence having a sequence identity of at least 70% with SEQ ID NO: 1, provided that said peptides do not have the amino acid sequence SEQ ID NO: 2 or SEQ ID NO: 3, and that the cysteine at position 6 of SEQ ID NO: 1 remains unchanged. The invention also relates to a pharmaceutical composition thereof and to its uses in the treatment of tumours, in particular, in the treatment of cancer and metastases.

The present invention is within the field of biomedicine. In particular, the present invention relates to peptides comprising the amino acid sequence SEQ ID NO:1, with the proviso that said peptides do not have the amino acid sequence SEQ ID NO: 2 or SEQ ID NO: 3, and a pharmaceutical composition thereof. Furthermore, the invention also relates to its uses in the treatment of tumours, in particular, in the treatment of cancer and metastases.

### BACKGROUND ART

The activity of the c-Src oncoprotein is particularly high in glioblastoma (Tabernero, 2022. Oncogene, 41(45): 4917-4928), the most aggressive primary brain tumour, which has a survival time of less than 2 years after diagnosis. Thanks to c-Src activity, glioblastoma stem cells, which are responsible for the recurrence and thus lethality of this tumour, are able to survive, proliferate, maintain the stem cell phenotype and invade adjacent healthy tissue. c-Src interacts with its ligands through its SH3 domain that binds to proline-rich regions within its target protein.

Connexin43 (Cx43) [NCBI accession number AAA52131, version AAA52131.1] is an integral membrane protein that is widely expressed in different mammalian tissues. In the central nervous system (CNS), Cx43 is primarily localised in astrocytes, the major type of glia, where it forms gap junctions (Gjs), intercellular communication channels that allow astrocytes to behave cooperatively. Since Lowenstein proposed in the 1960s that Gjs regulate cell proliferation, the role of Gjs and connexins in the regulation of cell proliferation has been extensively studied.

Application WO2014191608 A1 describes the development of a cell-penetrating peptide containing the 266-283 sequence of connexin-43 (Cx43) fused to the TAT penetrating sequence (TAT-Cx43₂₆₆₋₂₈₃). Application WO2014191608 A1 demonstrates that TAT-Cx43₂₆₆₋₂₈₃, which comprises the canonical SH3-binding region, binds to c-Src and to its endogenous inhibitors Csk and PTEN. Binding of c-Src to its inhibitors in this region leads to the inhibition of c-Src activity (González-Sánchez A. et al., Oncotarget, 7(31):49819-49833 (2016); Jaraiz-Rodriguez M. et al., J Vis Exp. (130):56457 (2017)) and its subsequent antitumour effect.

Furthermore, TAT-Cx43₂₆₆₋₂₈₃, like Cx43, is able to inhibit c-Src activity and the protumoral processes that depend on the oncogenic activity of c-Src (Herrero-González, S. et al., Oncogene, 29(42):5712-23 (2010); Gangoso, E. et al., Cell Death Dis. 5(1):e1023 (2014)), thus inducing a potent antitumour effect in human and mouse glioblastoma stem cells (Jaraiz-Rodriguez, M. et al., Stem Cell Reports 9, 451-463 (2017)), both in vitro and in vivo, without affecting healthy brain cells (Jaraiz-Rodriguez, M. et al., Neuro-Oncology, 22, 493-504 (2020)).

However, in the development of molecules with therapeutic potential, an important parameter to take into account is their size, since, in general, a larger size is related to a worse membrane permeability, metabolic instability, higher undesired interactions, higher side effects, lower specificity, higher costs or worse bioavailability. Thus, in view of the above, there is a need in the state of the art to provide alternative compounds of smaller size than the existing ones, with antitumour effect and/or capable of reversing the phenotype of tumour stem cells.

### DESCRIPTION OF THE INVENTION

As explained in the background, it is known that connexin-43 (Cx43) reverses the phenotype of human glioma stem cells and that this effect resides in a portion of the carboxyl-terminal end of Cx43.

In the present invention, the inventors have identified that even shorter amino acid sequences of Cx43 are able to maintain, and even slightly enhance, the anti-tumour effect in tumour stem cells. In particular, the inventors have shown that the sequence 266-275 of Cx43 (SEQ ID NO: 1), and peptides comprising such sequence, such as peptides comprising the sequences SEQ ID NO: 4, SEQ ID NO: 5 and SEQ ID NO: 6, reduce cell proliferation and survival (FIGS. 5 and 6), as well as cell migration (FIG. 7) of tumour stem cells, such as glioblastoma stem cells. The inventors found that the sequence 266-275 of Cx43 (SEQ ID NO: 1) is sufficient to bind to c-Src and to its endogenous inhibitor Csk (FIG. 9). Furthermore, the sequence 266-275 (SEQ ID NO: 1) is able to bind to albumin, which protects the peptide from proteases and prevents a rapid renal clearance (FIG. 8). Without intending to be linked to any theory, it is believed that cysteine at position 271 is required for intracellular interactions and albumin binding, presumably through a disulfide bond.

The amino acid sequences of the peptides of the invention maintain and even slightly increase their antitumoural effect, despite the fact that with respect to SEQ ID NO: 3 (Cx43₂₆₆₋₂₈₃), residues belonging to the canonical proline-rich region of Cx43, comprised between amino acids 274-283 and responsible for binding to the SH3 domain of c-Src, have been eliminated.

These SH3 domains (Src homology 3) are involved in the regulation of important cellular pathways, such as cell proliferation or migration, processes whose deregulation is closely related to tumour development. The binding of c-Src to its inhibitors in the region 266-283 from Cx43 leads to the inhibition of c-Src activity and its consequent antitumour effect. However, although previous structural studies indicated that the canonical SH3 domain-binding region of c-Src in Cx43 (274-283) was key to the antitumour effect of TAT-Cx43₂₆₆₋₂₈₃, the inventors have shown that the antitumour effect resides in the sequence between amino acids 266-275 of Cx43 (SEQ ID NO: 1). In fact, the inventors found that the sequence 266-275 of Cx43 (SEQ ID NO: 1) is sufficient to bind to c-Src and its endogenous inhibitor Csk (FIG. 9). Further, the results show that cysteine 271 is required for this antitumour effect (FIGS. 1 and 9).

The development of peptides with shorter amino acid sequences, which have antiproliferative and cell migration reduction capacity (as in the case of the peptides of the present invention), presents as associated advantages a higher solubilisation, simpler folding and improved cell internalisation, favouring their administration in the treatment of diseases that involve cell proliferation or in which tumour stem cells are involved, such as cancer, including metastatic cancer. In addition, having shorter peptides prevents the appearance of undesired effects due to interaction of the rest of Cx43 with other cellular effectors. Finally, an added advantage associated with the use of peptides of the present invention is that it is possible to attach cell internalisation sequences to the peptides so that, when administered to a subject, they can be internalised into the cell to exert their effect without the need for gene therapy techniques.

Based on this, the inventors have developed a number of inventive aspects which will be described below:

### Peptides of the Invention

An aspect of the invention relates to a peptide comprising, or consisting of, the amino acid sequence having a sequence identity of at least 70, 75, 80, 85, 90, 91, 92, 93, 94, 95, 96, 97, 98 or 99% with SEQ ID NO: 1 (Cx43₂₆₆₋₂₇₅) [AYFNGCSSPT], hereinafter the “peptide of the invention”, with the proviso that said peptide has neither the amino acid sequence SEQ ID NO: 2 (the carboxyl-terminal end of Cx43) nor SEQ ID NO: 3 (Cx43₂₆₆₋₂₈₃; peptide described in WO2014191608 A1), and that the cysteine at position 6 of SEQ ID NO: 1 (C₂₇₁) remains unchanged.

The peptide of the invention may comprise in its amino acid sequence conservative amino acid substitutions that maintain the ability of the peptide to reverse the tumour stem cell phenotype and/or to inhibit cell proliferation, in particular to inhibit the proliferation of a tumour stem cell. Therefore, in the context of the present invention, peptides derived from the peptide of the invention, so-called “variants”, are also encompassed within the term “peptide of the invention”. These variants, although they do not have 100% sequence identity with the peptide of the invention, retain their ability to inhibit cell proliferation as the amino acids have been replaced by biologically similar ones. Tests to ascertain whether a peptide derived from the peptide of the invention has the ability to reverse the phenotype of a tumour cell, inhibit cell proliferation or reduce cell migration are described in the Examples section. Thus, the peptide of the invention comprises, or consists of, an amino acid sequence having a sequence identity of at least 70, 75, 80, 85, 90, 91, 92, 93, 94, 95, 96, 97, 98 or 99% with SEQ ID NO: 1, with the proviso that said peptide has neither the amino acid sequence SEQ ID NO: 2 nor SEQ ID NO: 3, and that the cysteine at position 6 of SEQ ID NO: 1 (C₂₇₁) remains unchanged. In a more particular embodiment of the peptide of the invention, the alanine at position 1 of SEQ ID NO: 1 (A₂₆₆), the Tyrosine at position 2 of SEQ ID NO: 1 (Y₂₆₇) and/or the phenylalanine at position 3 of SEQ ID NO: 1 (F₂₆₈) remain unchanged. In a particular embodiment, the peptide of the invention comprises, or consists of, an amino acid sequence having a sequence identity of 100% with SEQ ID NO: 1.

As use herein, the term “sequence identity” or “identity” refers to the degree of similarity between two nucleotide or amino acid sequences obtained by aligning the two sequences, i.e.: the proportion of identical nucleotide or amino acids between two compared nucleotide sequences or polypeptides/proteins along their full-length sequence. Depending on the number of common residues between the aligned sequences, a different degree of identity, expressed as a percentage, will be obtained. The degree of identity between two amino acids or nucleotides sequences may be determined by conventional methods, for example, by standard sequence alignment algorithms known in the state of the art, such as BLAST [Altschul S. F. et al. Basic local alignment search tool. J Mol Biol. 1990 Oct. 5; 215(3): 403-10]. The BLAST programmes, for example, BLASTN, BLASTX, and T BLASTX, BLASTP and TBLASTN, are in the public domain at The National Center for Biotechonology Information (NCBI) website. The skilled person in the art understands that mutations in the nucleotide sequence of genes that lead to conservative amino acid substitutions at non-critical positions for the functionality of the protein are evolutionarily neutral mutations which do not affect its global structure or its functionality.

SEQ ID NO: 2 shows the amino acid sequence, and corresponds to, the carboxyl-terminal end of Cx43 (Cx43CT):

SPSKDCGSPKYAYFNGCSSPTAPLSPMSPPGYKLVTGDRNNSSCRNYNK
QASEQNWANYSAEQNRMGQAGSTISNSHAQPFDFPDDNQNAKKVAAGHE
LQPLAIVDQRPSSRASSRASSRPRPDDLEI

SEQ ID NO: 3 shows the amino acid sequence, and corresponds to, peptide Cx43₂₆₆₋₂₈₃ described in WO2014191608 A1:

AYFNGCSSPTAPLSPMSP.

As previously explained, any peptide comprising or containing in its amino acid sequence SEQ ID NO: 1 (Cx43₂se-27s), has the ability to reverse the phenotype of a tumour stem cell and/or to inhibit cell proliferation, in particular, to inhibit cell proliferation of a tumour stem cell, as well as to reduce cell migration.

The inventors have further experimentally demonstrated that other peptides comprising SEQ ID NO: 1 also exhibit this effect. Thus, in a particular embodiment, the peptide of the invention comprises, or consists of, the amino acid sequence SEQ ID NO: 4 (Cx43₂₆₆₋₂₇₇) [AYFNGCSSPTAP].

In another particular embodiment, the peptide of the invention comprises, or consists of, the amino acid sequence SEQ ID NO: 5 (Cx43₂₆₆₋₂₇₉) [AYFNGCSSPTAPLS].

In another particular embodiment, the peptide of the invention comprises, or consists of, the amino acid sequence SEQ ID NO: 6 (Cx43₂₆₆₋₂₈₁) [AYFNGCSSPTAPLSPM].

In the context of the present invention, a peptide is understood to be a molecule formed by joining between 6 and 150 amino acids by means of peptide bonds. However, in a particular embodiment, alone or in combination with the previous particular embodiments, the peptide of the invention has a length of from 10 to 100 amino acids (including the extremes), more particularly from 10 to 50 amino acids (including the extremes), from 10 to 40 amino acids (including the extremes), from 10 to 30 amino acids (including the extremes) or from 10 to 20 amino acids (including the extremes). In a still more particular embodiment, the peptide of the invention has a length of 10 to 16 amino acids (including the extremes). As an expert in the field understands, the smaller the peptide, the easier it is to administer and the fewer side effects it has.

The peptide of the invention can be obtained by techniques widely known in the state of the art. Examples of techniques for obtaining peptides include, but are not limited to, chemical or biological synthesis, genetic recombination or expression of polynucleotides coding for the peptide of the invention.

Additionally, the carboxyl and amino terminal ends of the peptide of the invention may be protected against proteolysis. For example, the amino-terminal end may be in the form of an acetyl group and/or the carboxyl-terminal end may be in the form of an amide group. It is also possible to carry out internal modifications of the peptides to make them resistant to proteolysis, for example, where at least one peptide bridge —CONH— is modified and replaced by a reduced bond (CH2NH), a retroinverse bond (NHCO), an oxymethylene bond (CH2-O), a thiomethylene bond (CH2-S), a ketomethylene bond (CO—CH2), a hydroxyethylene bond (CHOH—CH2), an (N—N) bond, an E-alkene bond or a —CH═CH— bond. The amino acids of the peptide of the invention can be in D-configuration, which can give rise to peptides resistant to proteolysis. Furthermore, in the present invention, it is contemplated that the peptides of the invention may be associated with protecting systems or molecular vehicles, including but not limited to, albumin. The peptides can also be stabilised by intramolecular cross-linking, for example, by modification of at least two amino acid residues with olefin side chains, preferably, C3-C8 alkenyl chains, preferably, pentel-2-yl chains, followed by cross-linking of the chains as described in the so-called “staple” technology (Walensky et al., 2004, Science 205: 1466-1470). All these peptides chemically modified to resist proteolysis are also covered by the present invention.

Additional modifications to the peptide of the invention comprise covalent bound to a polyethylene glycol (PEG) molecule at its carboxyl-terminal end or to a lysine residue, in order to decrease its urinary elimination and therapeutic dose, and to increase the half-life of the peptide in blood plasma. The half-life of the peptide can also be increased by embedding the peptide in a biodegradable and biocompatible polymeric material to form microspheres that are used as a drug delivery system. Polymers and copolymers are, for example, poly(D,L-lactide-co-glycolic acid) or PLGA. The techniques and procedures of how to manufacture lipid microspheres or nanocapsules for use in drug delivery are widely known to the skilled person in the art. The peptides of the invention may be associated with albumin or other systems that decrease their urinary elimination, increase the half-life and/or improve the pharmacokinetic profile. Any other method of pharmacological administration to selectively target a tumour population can be employed in the context of the present invention.

Furthermore, the use of biotin in the distribution and/or targeting of molecules to tumour cells is well known in the state of the art because biotin receptors are overexpressed in tumour cells. Therefore, the binding of molecules, such as peptides, to biotin improves their distribution or targeting to tumour cells. Thus, in a preferred embodiment, alone or in combination with all or each one of the previous particular embodiments, the peptide of the invention is covalently bound to biotin.

As the expert of the field understands, in order for the peptide of the invention to reverse the stem cell phenotype and/or inhibit cell proliferation, it is necessary for it to enter the cell and interact with the corresponding molecules, such as, for example, the tyrosine kinase c-Src, its active form Y416-Src, or its endogenous inhibitor, Csk. Introduction of the peptide into the cell can be done by any of the procedures known in the state of the art, including, but not limited to, direct injection, electroporation or transfection. However, these are relatively complex techniques and have limitations to be applied in vivo an access the entire tumour cell population. In case the peptide is to be administered to a subject, the peptide can be introduced into the cell by gene therapy techniques using viral vectors, or by cell internalisation sequences that allow the peptide to cross the plasma membrane.

Thus, in a particular embodiment, the peptide of the invention is covalently bound to a cell internalising amino acid sequence.

In the present invention, “cell internalising amino acid sequence” or “cell internalising sequence” or “cell penetrating peptides” (CPPs) refers to amino acid sequences that possess the ability to transport molecules across the plasma membrane without loss of integrity. Among the most commonly used sequences are TAT, Antennapedia (Antp) and oligo-arginines, whose common feature is the presence of cationic amino acid groups. These internalisation sequences allow internalisation of the peptide directly into the cell.

Examples of cell internalisation sequences include, but are not limited to, RQIKIWFQNRRMKWKK (SEQ ID NO: 12), VKKKKIKREIKI (SEQ ID NO: 13) [Guergnon J, et al. 2006. Mol Pharmacol. 69(4): 1115-24], FFLIPKG (SEQ ID NO: 14) [Ueda et al. 2012. Biomaterials, 35: 9061], SMoCs [Okuyama et al., 2007. Nature Methods, 4, 153-159], YGRKKKRRQRRR (SEQ ID NO: 7), DSLKSYWYLQKFSWR (SEQ ID NO: 15), KLWMRWWSPTTRRYG (SEQ ID NO: 16), RLWMRWYSPWTRRWG (SEQ ID NO: 17), RLIMRIYAPTTRRYG (SEQ ID NO: 18), RLYMRYYSPTTRRYG (SEQ ID NO: 19), RLWMRWYSPRTRAYG (SEQ ID NO: 20), KRPTMRFRYTWNPMK (SEQ ID NO: 21), WKCRRQCFRVLHHWN (SEQ ID NO: 22), WKCRRQAFRVLHHWN (SEQ ID NO: 23), WKARRQAFRVLHHWN (SEQ ID NO: 24), the penetrant sequence in glioma cells (Berges et al. Plos One 2012; 7(11):e49436) and Biotin-YSSYSAPVSSSLSVRRSYSSSSGS-CONH2 (SEQ ID NO: 25).

In a particular embodiment of the peptide of the invention, alone or in combination with the all or each one of the previous particular embodiments, the cell internalisation sequence comprises, or consists of, the amino acid sequence SEQ ID NO: 7 (YGRKKRRQRRR). Throughout this document, the term “TAT sequence” or “TAT” is also used to refer to SEQ ID NO: 7.

The cell internalisation sequence can be attached to the peptide of the invention at either the amino-terminal or the carboxyl-terminal end of the peptide. However, in a particular embodiment of the peptide of the invention, alone or in combination with the all or each one of the previous particular embodiments, the cell internalisation sequence is attached to the amino-terminal end of the peptide.

Thus, in the present invention, the peptides SEQ ID NO: 1, SEQ ID NO: 4, SEQ ID NO: 5 and SEQ ID NO: 6, can be found attached to a cell internalisation sequence.

In a particular embodiment, the peptide of the invention comprises, or consists of, the amino acid sequence SEQ ID NO: 8, SEQ ID NO: 9, SEQ ID NO: 10 or SEQ ID NO: 11.

SEQ ID NO: 8
-YGRKKRRQRRRAYFNGCSSPT
SEQ ID NO: 9
-YGRKKRRQRRRAYFNGCSSPTAP
SEQ ID NO: 10
-YGRKKRRQRRRAYFNGCSSPTAPLS
SEQ ID NO: 11
-YGRKKRRQRRRAYFNGCSSPTAPLSPM

As explained above, in the present invention we refer to “peptide of the invention” as a peptide comprising, or consisting of, the amino acid sequence having a sequence identity of at least 70, 75, 80, 85, 90, 91, 92, 93, 94, 95, 96, 97, 98 or 99% with SEQ ID NO: 1, with the proviso that such peptide does not have amino acid sequence SEQ ID NO: 2 or SEQ ID NO: 3, and that the cysteine at position 6 of SEQ ID NO: 1 (C₂₇₁) remains unchanged. Thus, a peptide comprising the amino acid sequence SEQ ID NO: 1, SEQ ID NO: 4, SEQ ID NO: 5, SEQ ID NO: 6, SEQ ID NO: 8, SEQ ID NO: 9, SEQ ID NO: 10 or SEQ ID NO: 11, as well as variants thereof, is encompassed under the term “peptide of the invention” or “peptides of the invention”, terms used interchangeably herein.

In a particular embodiment, alone or in combination with all the each one of the previous particular embodiments, variants of the peptide of the invention comprise, or consist of, an amino acid sequence having a sequence identity of at least 70, 75, 80, 85, 90, 91, 92, 93, 94, 95, 96, 97, 98 or 99% with SEQ ID NO: 4, SEQ ID NO: 5, SEQ ID NO: 6, SEQ ID NO: 8, SEQ ID NO: 9, SEQ ID NO: 10 or SEQ ID NO: 11. In another particular embodiment, alone or in combination with all or each one of the previous particular embodiments, the peptide of the invention comprises, or consists of, an amino acid sequence having a sequence identity of 100% with SEQ ID NO: 4, SEQ ID NO: 5, SEQ ID NO: 6, SEQ ID NO: 8, SEQ ID NO: 9, SEQ ID NO: 10 or SEQ ID NO: 11. The term “sequence identity” has been defined in previous paragraphs.

As previously indicated, the peptide of the invention can be obtained by techniques widely known in the state of the art, such as the expression in a cell of the polynucleotide encoding the peptide of the invention, and its subsequent isolation. Thus, in another aspect, the invention relates to a polynucleotide, hereinafter the “polynucleotide of the invention”, encoding the peptide of the invention or a variant thereof as defined previously.

The term “polynucleotide”, as used in the present invention, refers to a polymeric form of nucleotides of any length, consisting of ribonucleotides and/or deoxyribonucleotides. The term includes both single and double stranded polynucleotides as well as modified (methylated, protected and the like) polynucleotides. The polynucleotide of the invention can be DNA, RNA or cDNA.

In another aspect, the invention relates to a gene construct, hereinafter the “gene construct of the invention”, comprising the polynucleotide of the invention.

Preferably, the gene construct comprises the polynucleotide of the invention operatively linked to regulatory sequences for expression of the polynucleotide of the invention. In principle, any promoter can be used in the gene constructs of the present invention, provided that such promoter is compatible with the cells in which it is desired to express the polynucleotide.

A promoter, or promoter region, is a sequence of nucleotides that controls the transcription of a given gene (nucleotide sequences). In the present invention it refers to a nucleotide sequence that controls the transcription of the polynucleotide of the invention. Promoter sequences can be unidirectional or bidirectional. A unidirectional promoter is one that controls the transcription of a gene or more genes that are placed in tandem with the first. “Tandem” means that the 3′ end of the first gene is followed, either consecutively or separated by a specific nucleotide sequence, by the 5 end of the second gene. A bidirectional promoter refers to the promoter region that controls transcription in two opposite directions, e.g. the sequence preceding the kivD gene of L. lactis IFPL730 which acts as a bidirectional promoter. That is, a bidirectional promoter directs the transcription of two genes located divergently, i.e. in opposite directions, the 5′ end of both nucleotide sequences being closer to each other than the 3′ end. In the present invention the terms “promoter” and “promoter region” are used interchangeably. Furthermore, promoters in the present invention may be constitutive or inducible. The term “inducible”, as used in the present description, refers to the possibility that the promoter has a control element that allows the transcription of the regulated gene to be turned on or off (repressed) in the presence of a factor external to the promoter.

Promoters suitable for the embodiment of the present invention include, without being limited to, constitutive promoters such as those derived from the genomes of eukaryotic viruses such as polyoma virus, adenovirus, SV40, CMV, avian sarcoma virus, hepatitis B virus, the metallothionein gene promoter, the thymidine kinase gene promoter of herpes simplex virus, LTR regions of retroviruses, the immunoglobulin gene promoter, the actin gene promoter, the EF-1 alpha gene promoter, the EF-1 alpha gene promoter as well as inducible promoters in which protein expression depends on the expression of the protein, the immunoglobulin gene promoter, the actin gene promoter, the EF-1 alpha gene promoter as well as inducible promoters where protein expression is dependent on the addition of an exogenous molecule or signal, such as the tetracycline system, the NFKB/VV light system, the Cre/Lox system and the promoter of heat shock genes, the RNA polymerase 11 regulatory promoters as well as tissue-specific promoters. Glial cell-specific gene promoters include GFAP, nestin and S-100. Glioma stem cell promoters include Id1 and Sox-2.

Additionally, the gene construct of the invention may comprise or contain markers or tags that allow the isolation of the peptide of the invention once it is synthesised in the cell.

On the other hand, the polynucleotide or gene construct of the invention may be forming part of a vector. Thus, in another aspect, the invention relates to a vector, hereinafter “vector of the invention”, comprising the polynucleotide or gene construct of the invention.

As understood by the skilled person in the art, the type of vector used may be a cloning vector suitable for propagation or an expression vector. Thus, examples of suitable vectors according to the present invention include, but are not limited to, expression vectors in prokaryotes such as pUC18, pUC19, Bluescript and its derivatives, mp18, mp19, pBR322, pMB9, ColEI, pCRI, RP4, phage and shuttle vectors such as pSA3 and pAT28, yeast expression vectors such as 2-micron plasmid type vectors, integration plasmids, YEP vectors, centromeric and similar plasmids, insect cell expression vectors such as pAC series vectors and pVL series vectors, plant expression vectors such as pIBI series vectors, pEarleyGate, pAVA, pCAMBIA, pGSA, pGWB, pMDC, pMY, pORE and similar and expression vectors in higher eukaryotic cells either based on viral vectors (adenoviruses, adenovirus-associated viruses as well as retroviruses and lentiviruses) as well as non-viral vectors such as pSilencer 4.1-CMV (Ambion), pcDNA3, pcDNA3.1/hyg pHCMV/Zeo, pCR3.1, pEFVHis, pIND/GS, pRc/HCMV2, pSV40/Ze02, pTRACER HCMV, pUB6N5-His, pVAXI, pZeoSV2, pCI, pSVL and pKSV-10, pBPV-1, pML2d and pTDTI.

In a particular embodiment, alone or in combination with all or each one of the previous embodiments, the vector of the invention is a viral vector, preferably, a viral vector of a retrovirus, a lentivirus or an adenovirus.

The vector of the invention can be used to transform, transfect or infect cells susceptible of being transformed, transfected or infected by said vector. Such cells may be prokaryotic or eukaryotic. By way of example, the vector into which the nucleotide sequence, preferably DNA, is introduced may be a plasmid or a vector which, when introduced into a host cell, is integrated into the genome of that cell and replicates together with the chromosome (or chromosomes) into which it (or they) is (are) integrated. Such a vector can be obtained by conventional methods known to a person skilled in the art.

Therefore, another aspect of the invention relates to a cell, hereinafter the “cell of the invention”, comprising the polynucleotide, gene construct or vector of the invention, for which such cell may have been transformed, transfected or infected with a construct or vector provided by this invention. Transformed, transfected or infected cells can be obtained by conventional methods known to those experts in the field. In a particular embodiment, a host cell is an animal cell transfected or infected with an appropriate vector.

Suitable host cells for expression of the peptide of the invention include, but are not limited to, mammalian, plant, insect, fungal and bacterial cells. Bacterial cells include, but are not limited to, Gram-positive bacterial cells such as Bacillus, Streptomyces and Staphylococcus species and Gram-negative bacterial cells such as Escherichia and Pseudomonas cells. Fungal cells preferably include yeast cells such as Saccharomyces, Pichia pastoris and Hansenula polymorpha. Insect cells include, but are not limited to, Drosophila cells and Sf9 cells. Plant cells include, but are not limited to, cells from crop plants such as cereals, medicinal, ornamental or bulb plants. Mammalian cells suitable for use in the present invention include epithelial cell lines (porcine, etc.), osteosarcoma cell lines (human, etc.), neuroblastoma cell lines (human, etc.), epithelial carcinomas (human, etc.), glial cells (murine, etc.), hepatic cell lines (monkey, etc.), CHO cells (Chinese Homo, etc.), liver cells (monkey, etc.), and Sf9 cells (human, etc.).), CHO (Chinese Hamster Ovary) cells, COS cells, BHK cells, HeLa cells, 911, AT1080, A549, 293 or PER.C6 cells, human ECCs 5 NTERA-2 cells, D3 cells of the mESCs line, human embryonic stem cells such as HS293 and BGV01, SHEF1, SHEF2 and HS181, NIH3T3, 293T, REH and MCF-7 cells and hMSCs.

The inventors have identified that the peptide of the invention is capable of reversing the phenotype of tumour stem cells and/or inhibiting cell proliferation, in particular inhibiting the proliferation of tumour stem cells, such as glioma stem cells, as well as reducing their cell migration. Furthermore, the peptide, polynucleotide, gene construct, vector or cell of the invention may be part of a composition, preferably a pharmaceutical composition, as an active drug or active ingredient.

Thus, another aspect of the invention relates to a composition, hereinafter, the “composition of the invention”, comprising the peptide, polynucleotide, gene construct, vector or cell of the invention. In a particular embodiment, the composition of the invention is a pharmaceutical composition.

In a particular embodiment, the composition of the invention comprises, or consist of, the peptide, polynucleotide, gene construct, vector or cell of the invention in a therapeutically effective amount, and a vehicle which, in case of a pharmaceutical composition, will be a pharmaceutically acceptable vehicle.

In the present invention, “pharmaceutical composition” means any pharmaceutical preparation or form, the formula of which, expressed in units of the international system, comprises a substance or mixture of substances, with constant weight, volume and percentages, manufactured in legally established pharmaceutical laboratories, packaged or labelled for distribution and marketed as effective for the diagnosis, treatment, mitigation and prophylaxis of a disease, physical anomaly or symptom, or the restoration, correction or modification of the balance of the organic functions of human beings and animals. The preparation of the pharmaceutical composition can be carried out by any of the methods described in the prior art.

In the sense of this description, the expression “effective dose” refers to the amount of the compound or pharmaceutical composition of the invention which will produce the desired effect and will generally be determined, inter alia, by the characteristics of the compound or pharmaceutical composition and the therapeutic effect to be achieved. The dosage to obtain a therapeutically effective amount depends on a variety of factors such as, for example, the age, weight, sex or tolerance of the mammal. Pharmaceutically acceptable adjuvants” and “pharmaceutically acceptable vehicles” which may be used in such compositions are vehicles known in the prior art.

The term “vehicle” refers to a diluent or excipient with which the active substance is administered. Such vehicles may be sterile liquids, such as water and oils, including those of petroleum, animal, vegetable or synthetic origin, such as peanut oil, soybean oil, mineral oil, sesame oil and similar. Water or aqueous solutions of saline and aqueous solutions of dextrose and glycerol are preferably used as vehicles, particularly for injectable solutions. Preferably, in the case of pharmaceutical vehicles, these are approved by a state or federal government regulatory agency or are listed in the US Pharmacopoeia or other generally recognised pharmacopoeia for use in animals, and more particularly in humans. The vehicles and auxiliary substances necessary to manufacture the desired pharmaceutical dosage form of administration of the pharmaceutical composition of the invention will depend, among other factors, on the chosen pharmaceutical dosage form of administration. Such pharmaceutical dosage forms for administration of the pharmaceutical composition will be manufactured according to conventional methods known to the person skilled in the art.

Furthermore, in the present invention, it is contemplated that the peptides of the invention may be associated with delivery systems or molecular vehicles, including but not limited to, exosomes and microvesicles. These delivery systems or molecular vehicles are capable by their properties of crossing cell membranes and delivering their “cargo”, in the present invention the peptides of the invention, in a biologically active form, and in addition, can cross biological membranes such as the blood-brain barrier. Thus, in a particular embodiment, alone or in combination with all or each one of the previous particular embodiments, the composition of the invention further comprises exosomes and/or microvesicles.

The compositions of the present invention can be formulated for administration to an animal, and more preferably to a mammal, including a human, in a variety of forms known in the prior art. Thus, they may be, without limitation, in aqueous or non-aqueous solutions, in emulsions or in suspensions. Examples of non-aqueous solutions are, for example, but without limitation, propylene glycol, polyethylene glycol, vegetable oils, such as olive oil, or injectable organic esters, such as ethyl oleate. Examples of aqueous solutions are, for example, but not limited to, water, alcoholic solutions in water, or saline media. Aqueous solutions may be buffered or unbuffered and may have additional active or inactive components. Additional components include salts to modulate ionic strength, preservatives including, but not limited to, antimicrobial agents, antioxidants, chelators, or the like, or nutrients, including glucose, dextrose, vitamins, and minerals. Alternatively, the compositions can be prepared for administration in solid form. The compositions may be combined with various vehicles or inert excipients, including but not limited to binders, such as microcrystalline cellulose, gum tragacanth, or gelatin; excipients, such as starch or lactose; dispersing agents, such as alginic acid or corn starch; lubricants, such as magnesium stearate, glidants such as colloidal silicon dioxide; sweetening agents, such as sucrose or saccharin; or flavouring agents, such as peppermint or methyl salicylate.

Additionally, the composition of the invention may comprise an adjuvant. “Adjuvant” means any substance which enhances the effectiveness of the pharmaceutical composition of the invention. Examples of adjuvants include, but are not limited to, adjuvants consisting of aluminium (alum) salts, such as aluminium hydroxide, aluminium phosphate or aluminium sulphate, oil-in-water or water-in-oil emulsion formulations such as Complete Freund's Adjuvant (ACF) as well as Incomplete Freund's Adjuvant (AIF), mineral gels, gels, block copolymers, Avridine™, SEAM62, adjuvants consisting of bacterial cell wall components such as adjuvants including liposaccharides (e.g. lipid A or Monophosphoryl Lipid A (MLA), trehalose dimycolate (TDM), and cell wall skeleton components (CWS), heat shock proteins or their derivatives, adjuvants derived from ADPribosylated bacterial toxins, including diphtheria toxin (DT), pertussis toxin (PT), cholera toxin (CT), the heat labile toxins of E. coli heat labile toxins (LT1 and LT2), Endotoxin A and Pseudomonas exotoxin, B. cereus exoenzyme B, B. sphaerieus toxin, C. botulinum toxins C2 and C3, C. limosum exoenzyme as well as the toxins of C. perfringens, C. spiriforma and C. diffielle, S. aureus, EDIM and toxin mutants such as CRM-197, non-toxic mutant diphtheria toxin; saponins such as ISCOMs (immunostimulatory complexes), chemokines and cytokines such as interleukins (IL-I IL-2, IL-4, IL-5, IL-6, IL-7, IL-8, IL-12, etc), interferons (such as interferon gamma), macrophage colony-stimulating factor (M-CSF), tumour necrosis factor (TNF), defensins 102, RANTES, MIPI-alpha, and MEP-2, muramyl peptides such as N-acetyl-muramyl-L-threonyl-D-isoglutamine (thr-MDP), N-acetyl normuramyl-L-alanyl-D-isoglutamine (nor-MDP), N-acetylmuramyl-L-alanyl-Disoglutaminyl-L-alanine-2-(1′-2′-dipalmitoyl-s-n-glycero-3 huydroxyphosphoryloxy)ethylamine (MTP-PE) etc; adjuvants derived from the CpG family of molecules, synthetic CpG dinucleotides and oligonucleotides comprising CpG motifs, lysosum exoenzyme from C. limosum and synthetic adjuvants such as PCPP, cholera toxin, Salmonella toxin, alum and similar, aluminium hydroxide, N-acetyl-muramyl-L-threonyl-D-isoglutamine (thr-MDP), N-acetyl-nor-muramyl-L-alanyl-D-isoglutamine, MTP-PE and RIBI, which contains three components extracted from bacteria, monophosphoryl lipid A, trehalose dimycolate and cell wall skeleton (MPL+TDM+CWS) in a 12%/Tween 80 squalene emulsion. Other examples of adjuvants include DDA (dimethyldioctadecylammonium bromide), complete and incomplete Freund's adjuvants, QuilA, microvesicles and exosomes.

The term “pharmaceutically acceptable” means that the vehicle or excipient must permit the activity of the compounds of the pharmaceutical composition, in particular of the peptide of the invention, i.e. be compatible with those components, so as not to cause harm to the organisms to which it is administered.

In a particular embodiment, alone or in combination with all or each one of the precious particular embodiments, the composition of the invention, more in particular, the pharmaceutical composition of the invention, further comprises a chemotherapeutic agent.

“Chemotherapeutic agent” means any substance that is capable of inhibiting cell proliferation without necessarily killing the cell, or that is capable of inducing cell death. Agents capable of inhibiting cell proliferation without causing cell death are generically termed cytostatic agents, whereas those capable of inducing cell death normally by activating apoptosis are generically termed cytotoxic agents. Non-limiting examples of chemotherapeutic agents suitable for use in the compositions of the invention include (i) microtubule stabilizing agents such as taxanes, paclitaxel, docetaxel, epothilones and laulimalides, (ii) kinase inhibitors such as Iressa®, Gleevec, Tarceva™, (Erlotinib HCl), BAY-43-9006, (iii) antibodies specific for receptors with kinase activity including, without limitation, Trastuzumab (Herceptin®), Erlotinib, Gefitinib, Cetuximab (Erbitux®), Bevacizumab (Avastin™), Rituximab (Ritusan®), Pertuzumab (Omnitarg™); (iv) mTOR pathway inhibitors, such as rapamycin and CCI-778; (v) Apo2L1Trail, (vi) anti-angiogenic agents such as endostatin, combrestatin, angiostatin, thrombospondin and vascular endothelial growth inhibitor (VEGI); (vii) antineoplastic vaccines including activated T-cell, non-specific immunopotentiating agents (e.g. interferons, interleukins) or other types of immunotherapy; (viii) antibiotic cytotoxic agents such as doxorubicin, bleomycin, dactinomycin, daunorubicin, epirubicin, mitomycin and mitozantrone; (ix) alkylating agents such as Melphalan, Carmustine, Lomustine, cyclophosphamide, ifosfamide, Chlorambucil, Fotemustine, Busulfan, Temozolomide and thiotepa; (x) antineoplastic hormonal agents such as Nilutamide, Cyproterone acetate, Anastrozole, Exemestane, Tamoxifen, Raloxifene, Bicalutamide, Aminoglutethimide, Leuprorelin acetate, Toremifene citrate, Letrozole, Flutamide, Megestrol acetate and Goserelin acetate; (xi) gonadal hormones such as cyproterone acetate and medoxyprogesterone acetate; (xii) anti-metabolites such as Cytarabine, Fluorouracil, Gemcitabine, Topotecan, Hydroxyurea, Thioguanine, Methotrexate, Colaspase, Raltitrexedo and Capicitabine; (xiii) anabolic agents such as nandrolone; (xiv) adrenal steroid hormones such as methylprednisolone acetate, dexamethasone, hydrocortisone, prednisolone and prednisone; (xv) antineoplastic agents such as Temozolamide, Carboplatin, Cisplatin, Oxaliplatin, Etoposide and Dacarbazine and (xvi) topoisomerase inhibitors such as topotecan and irinotecan.

Such compositions and/or their formulations can be administered to an animal, including a mammal and thus a human being, in a variety of forms, including, but not limited to, intraperitoneal, intravenous, intramuscular, subcutaneous, intrathecal, intraventricular, intra-articular, intratumoral, oral, enteral, parenteral, intranasal, ocular or topical. A preferred route of administration of the compositions and/or formulations of the compound of the invention for the prevention or treatment of a cancer is the intratumoral and intraperitoneal route. In a particular embodiment, alone or in combination with all or each one of the previous particular embodiments, the composition of the invention is formulated for oral, parenteral, nasal, sublingual or intratumoral administration.

### Therapeutic Uses of the Invention

As previously explained, the peptide of the invention, as well as the associated aspects thereof, the polynucleotide, the gene construct, the vector, the cell of the invention, are capable of reversing the phenotype of tumour stem cells and/or inhibiting cell proliferation, in particular inhibiting the proliferation of tumour stem cells, such as glioma stem cells, as well as reducing their cell migration. Likewise, the peptide, polynucleotide, gene construct, vector or cell of the invention may be part of a composition, preferably a pharmaceutical composition, having application in the treatment of tumours or cancer present in a subject.

In the present invention “subject” means any animal, preferably a mammal, more preferably a primate, in particular a human being, of any breed, sex or age.

Thus, another aspect of the invention relates to the peptide, polynucleotide, gene construct, vector, cell or composition of the invention for use as a medicament.

The term “medicament” as used herein refers to any composition/substance used for the prevention, diagnosis, alleviation, treatment or cure of disease in a subject or which may be administered to a subject for the purpose of restoring, correcting or modifying physiological functions by exerting a pharmacological, immunological or metabolic action.

In another aspect, the invention relates to the peptide, polynucleotide, gene construct, vector, cell or composition of the invention for use in the treatment of benign or malignant tumours.

In the present invention, “treatment” (or “treat” or “treating”) refers to processes involving a slowing, interrupting, arresting, controlling, stopping, reducing, or reversing the progression or severity of an existing symptom, disorder, condition, or disease, but does not necessarily involve a total elimination of all disease-related symptoms, conditions, or disorders. The treatment of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms, such as to lead to a halt in the progression of the disorder or disease or a delay in the progression of the disorder or disease) or complete response (e.g., disappearance of symptoms) of the subject/patient suffering from the disorder or disease. Such a partial or complete response may be followed by a relapse. It is to be understood that a subject/patient may experience a broad range of responses to a treatment (such as the exemplary responses as described herein above). In the context of the present invention, the disease or disorder is benign or malignant tumours, such as cancer.

In the present invention “tumour” means any growth of tissue generated by uncontrolled proliferation of cells. A tumour may be benign or malignant. A tumour is considered benign when the cells forming the tumour do not invade other tissues or metastasise to other parts of the body. Normally, a benign tumour is well encapsulated and the cells do not show changes in structure. In contrast, a tumour is considered malignant when the tumour cells can grow rapidly, show anaplasia and/or are able to infiltrate the tissue, invade adjacent tissues or even spread to other parts of the body, a process known as metastasis. In general, malignant tumours are known as cancer. Thus, in one particular embodiment, the malignant tumour is cancer which, in another more particular embodiment, the cancer is a metastatic cancer.

On the other hand, the tumour or cancer (if malignant) can be located or originate in any tissue or organ of the body. Thus, any tumour or cancer is susceptible to be treated with the pharmaceutical composition of the invention, regardless of its stage of development, its origin or its location. Examples of cancer include, but are not limited to, lung cancer, colon cancer, skin cancer, pancreatic cancer, stomach cancer, breast cancer, prostate cancer, liver cancer or cervical cancer. However, in a particular embodiment, the tumour (benign or malignant) is a Central Nervous System (CNS) tumour (i.e. it is located in the CNS), in a more particular embodiment, the tumour is a brain tumour, or a spinal cord tumour (benign or malignant).

In another particular embodiment, the tumour is a glioma. As used here, the term “glioma” refers to a type of neoplasm that occurs in the brain or spinal cord. It is called a glioma because it arises from glial cells. Its most frequent location is the brain, although it can also occur in the spinal cord. In the present invention, the composition of the invention can be used for the treatment of any glioma.

The new 2021 WHO Classification of Tumors of the Central Nervous System considers molecular alterations at least as important as histopathological features for glioma classification. Gliomas can be named according to the specific cell type they most closely resemble, such as ependymomas (ependymal cells), astrocytomas (astrocytes) or oligodendrogliomas (oligodendrocytes), and graded from 1 to 4 according to their malignancy degree, being 4 the most malignant form. Thus, in a particular embodiment, the glioma is a glioma selected from the group consisting of astrocytoma, oligodendroglioma, glioblastoma and ependymoma. In an even more particular embodiment, the tumour is glioblastoma, the most aggressive (grade 4) and frequent primary brain tumour.

It is known in the state of the art that tumour or cancer stem cells, in addition to possessing the typical properties of a stem cell, i.e., self-renewal and the ability to differentiate into multiple cell types, are resistant to conventional treatments and persist in tumours as a distinct population, causing tumour relapse and metastasis by growing or giving rise to new tumours. Thus, in a particular embodiment, alone or in combination with all the each one of the previous particular embodiments, the tumour comprises stem cells, preferably, tumour stem cells.

In a still more particular embodiment, the tumour stem cells are glioma stem cells, more particularly, the glioma stem cells are astrocytoma stem cells, oligodendroglioma stem cells, glioblastoma stem cells or ependymoma stem cells. Examples of glioblastoma stem cells include, but are not limited to, G166 cells, G179 cells, GIiNS2 cells or G144 cells (Pollard S. M. et al., Cell Stem Cell, 4, 568-580 (2009)), in general primary glioma stem cells obtained from glioblastomas of patients immediately after surgery, or murine glioblastoma stem cells. Thus, in a particular embodiment, the glioblastoma stem cells are selected from the list consisting of G166 cells, G179 cells, GIiNS2 cells, G144 cells, or other primary human glioblastoma stem cells. Preferably, the glioblastoma stem cells are G166 cells.

As previously explained, the peptide of the invention, as well as associated aspects thereof (the polynucleotide, polynucleotide, gene construct, vector or cell of the invention) which may form part of and be the active agent or active ingredient of a composition, preferably a pharmaceutical composition, are capable of reversing the phenotype of tumour stem cells and/or inhibiting cell proliferation, in particular inhibiting the proliferation of tumour stem cells, such as glioma stem cells, as well as reducing their cell migration.

Thus, one aspect of the invention relates to the peptide, polynucleotide, gene construct, vector, cell or composition of the invention for use in reversing the phenotype of tumour stem cells and/or inhibiting cell proliferation, in particular, inhibiting the proliferation of stem cells, more specifically, tumour stem cells, even more specifically, glioma stem cells. In another particular embodiment, the glioma stem cells are astrocytoma stem cells, oligodendroglioma stem cells, glioblastoma stem cells or ependymoma stem cells

Furthermore, as shown in the examples illustrating the present invention, the peptide of the invention reduces the migration of human glioma stem cells. Migration of tumour cells is the cause of invasion of other surrounding tissues or other parts of the body (metastasis), increasing the chances that they will escape surgery and cause recurrence of these tumours.

Thus, another aspect of the invention relates to the peptide, polynucleotide, gene construct, vector, cell or composition of the invention for use in preventing proliferation, migration and/or metastasis of tumour cells. In a particular embodiment, the tumour cells are stem cells. In another still more particular embodiment, the stem cells are glioma stem cells. In another particular embodiment, the glioma stem cells are astrocytoma stem cells, oligodendroglioma stem cells, glioblastoma stem cells or ependymoma stem cells.

In a particular embodiment, the glioblastoma stem cells are selected from the list consisting of G166 cells, G179 cells, GIiNS2 cells and G144 cells. Preferably, the glioblastoma stem cells are G166 cells.

### Uses of the Peptide of the Invention, and Aspects Derived Thereof, in the Manufacture of a Medicament.

The inventors have shown that the peptide of the invention is capable of reversing the phenotype of tumour stem cells and/or inhibiting cell proliferation, in particular inhibiting the proliferation of tumour stem cells, such as glioma stem cells, as well as reducing their cell migration. This allows the use of the peptide of the invention in the treatment of tumours or cancer present in a subject.

Thus, in another aspect, the invention relates to the use of the peptide, polynucleotide, gene construct, vector, cell or composition of the invention in the preparation/manufacture of a medicament/pharmaceutical composition. Techniques and procedures for making pharmaceutical compositions have been described previously herein.

In another aspect, the invention relates to the use of the peptide, polynucleotide, gene construct, vector, cell or composition of the invention in the preparation of a medicament/pharmaceutical composition for the treatment of benign or malignant tumours.

The terms “treatment”, “tumour”, “benign tumour”, or “malignant tumour” have already been defined or explained previously, and both they and their preferred embodiments are applicable to the present aspect of the invention.

Thus, in one particular embodiment, the malignant tumour is cancer which, in another more particular embodiment, is metastatic cancer.

In a particular embodiment, the tumour (benign or malignant) is a Central Nervous System (CNS) tumour (i.e. it is located in the CNS), in a more particular embodiment, the tumour is a brain tumour, or a spinal cord tumour (benign or malignant).

In another particular embodiment, the tumour is a glioma. In a more particular embodiment, the glioma is a glioma selected from the group consisting of astrocytoma, oligodendroglioma, glioblastoma, and ependymoma. Even more particularly, the glioblastoma is a glioblastoma multiforme.

In another particular embodiment, the tumour comprises stem cells, preferably tumour stem cells, more preferably, glioma stem cells, even more preferable, the glioma stem cells are astrocytoma stem cells, oligodendroglioma stem cells, glioblastoma stem cells or ependymoma stem cells.

As previously explained, the peptide of the invention, as well as associated aspects thereof, the polynucleotide, polynucleotide, gene construct, vector, cell or the composition of the invention, is capable of reversing the phenotype of tumour stem cells and/or inhibiting cell proliferation, in particular the proliferation of tumour stem cells, more in particular glioma stem cells, even more in particular, astrocytoma stem cells, oligodendroglioma stem cells, glioblastoma stem cells or ependymoma stem cells.

Thus, in another aspect, the invention relates to the use of the peptide, polynucleotide, gene construct, vector or cell of the invention in the preparation/manufacture of a medicament/pharmaceutical composition for reversing the phenotype of tumour stem cells and/or inhibiting cell proliferation, specifically, inhibiting the proliferation of stem cells, more specifically, tumour stem cells, and even more specifically, glioma stem cells, even still more specifically, the glioma stem cells are astrocytoma stem cells, oligodendroglioma stem cells, glioblastoma stem cells or ependymoma stem cells.

Furthermore, as shown in the examples illustrating the present invention, the peptide of the invention reduces the proliferation and migration of human glioma stem cells. Thus, another aspect of the invention relates to the use of the peptide, polynucleotide, gene construct, vector, cell or composition of the invention in the preparation/manufacture of a medicament/pharmaceutical composition for preventing the proliferation, migration and/or metastasis of tumour cells. In a particular embodiment, the tumour cells are stem cells. In another still more particular embodiment, the stem cells are glioma stem cells. Even in a still more particular embodiment, the glioma stem cells are astrocytoma stem cells, oligodendroglioma stem cells, glioblastoma stem cells or ependymoma stem cells.

### In Vitro Uses of the Peptide of the Invention

In addition to the therapeutic applications mentioned above, the application of the peptide of the invention, as well as the inventive aspects derived from it, in in vitro assays is also possible. Thus, another inventive aspect relates to the use of the peptide, polynucleotide, gene construct, vector, cell or composition of the invention to inhibit in vitro cell proliferation and/or to reverse in vitro the phenotype of tumour stem cells. In a particular embodiment, the tumour stem cells are glioma tumour stem cells. In a more particular embodiment, the glioma tumour stem cells are astrocytoma stem cells, oligodendroglioma stem cells, glioblastoma stem cells or ependymoma stem cells.

In the present invention, “reversing the phenotype of tumour stem cells” means that tumour stem cells lose their phenotypic characteristics, i.e. their high tumourigenic potential (ability to generate malignant tumours) and their resistance to conventional therapies in the treatment of tumours. Molecularly, a tumour stem cell is characterised by high expression of Id1, Sox2 and N-cadherin, low expression of Cx43 and E-cadherin, and high activity of c-Src. Thus, in the present invention it is said that “a peptide has the ability to reverse the phenotype of tumour stem cells” when said peptide upon contact with tumour stem cells causes said cells to lose the phenotypic characteristics mentioned. An example of an assay to check whether the phenotype of a tumour stem cell has been reversed is, for example, an immunocytochemical, PCR or western blot study of the expression of Id1, Sox2, N-cadherin, E-cadherin or any other stem cell marker.

In the present invention “inhibition of cell proliferation” means the reduction, decrease, attenuation or blockage of cell division or cell cycle. An assay to check that the cell proliferation of a cell has been inhibited is, for example, the MTT colorimetric assay.

In another aspect, the invention relates to the use of the peptide, polynucleotide, gene construct, vector, cell or composition of the invention for the in vitro identification of compounds that regulate or modulate tumourigenicity.

In a particular embodiment of the in vitro uses of the peptide of the invention, alone or in combination with all or each one of the previous embodiments, the concentration of the peptide of the invention is from 1 to 220 μM. In another more particular embodiment, the concentration of the peptide of the invention is from 10 to 200 μM. In yet another even more particular embodiment, the concentration of the peptide of the invention is from 40 to 60 μM (including the values 40 and 60 μM). More particularly, the concentration of the peptide of the invention is from 45 to 55 μM (including the values 45 and 55 μM). Still more particularly, the concentration of the peptide of the invention is 46, 47, 48, 49, 49, 50, 51, 52, 53, 54 or 55 μM. Preferably, the concentration of the peptide is 50 μM.

### Kit of the Invention and its Uses

Administration of the peptide, polynucleotide, gene construct, vector, cell or pharmaceutical composition of the invention may require a number of components, which in turn may be arranged together in the form of a kit.

Thus, in another aspect, the invention relates to a kit, hereinafter the “kit of the invention”, comprising the peptide, polynucleotide, gene construct, vector, cell or composition of the invention.

Components useful for administration and which may be comprised in the kit include, but are not limited to, buffer solution, lysis solution, sterile material (such as syringes, swabs or forceps), distilled water or alcohols (ethanol). Additionally, the kit may contain instructions or directions to guide the person skilled in its administration.

The kit of the invention, which has utility for use in the administration of the peptide, polynucleotide, gene construct, vector, cell or composition of the invention, can also be used in in vitro assays.

Thus, another aspect of the invention relates to the use of the kit of the invention for the in vitro determination of the effect of the peptide on the tumourigenicity of a cell line, to inhibit in vitro cell proliferation and/or to reverse the phenotype of tumour stem cells.

In a particular embodiment of the kit of the invention, inhibiting cell proliferation comprises inhibiting cell proliferation of tumour stem cells, preferably glioma stem cells, more preferably, the glioma stem cells are astrocytoma stem cells, oligodendroglioma stem cells, glioblastoma stem cells or ependymoma stem cells.

The terms used to define the kit and uses of the kit of the invention have already been explained, and both they and their preferred embodiments are applicable to the different uses of the kit of the invention

### Method of Treatment/Prevention of the Invention

In another aspect, the invention relates to a method for the treatment and/or prevention of tumours in a subject, both benign and malignant (cancer), comprising administering to said subject the peptide, polynucleotide, gene construct, vector, cell, or composition of the invention.

Another aspect of the invention relates to a method for the prevention of proliferation, migration and/or metastasis of tumour cells in a subject, comprising administering to said subject the peptide, polynucleotide, gene construct, vector, cell, or composition of the invention.

The terms defined and explained for the other aspects of the invention, as well as its preferred embodiments are also applicable to the treatment and/or prevention method of the invention.

### DESCRIPTION OF THE DRAWINGS

FIG. 1. Effect of cysteine 271 of the Cx43 sequence 266-283 on the survival and proliferation of human glioblastoma stem cells. G166 human glioblastoma stem cells were treated in the absence (control) or presence of TAT-Cx43₂₆₆₋₂₈₃ or the same peptide in which cysteine 271 was replaced by alanine (TAT-Cx43₂₆₆₋₂₈₃ C/A), both at a concentration of 50 μM. At 48h after the start of treatment, photomicrographs were taken, and cell viability was analysed by MTT assay. Results are expressed as percentage of control and are means±SEM (n=3). Significance of differences from control is expressed as ***p<0.001 (ANOVA).

FIG. 2. Comparison of the effect of TAT-Cx43₂₇₁₋₂₈₇ versus TAT-Cx43₂₆₆₋₂₈₃ on the survival and proliferation of human glioblastoma stem cells. G166 human glioblastoma stem cells were treated in the absence (control) or presence of TAT-Cx43₂₆₆₋₂₈₃ or TAT-Cx43₂₇₁₋₂₈₇, both at a concentration of 50 μM. At 72h after the start of treatment, photomicrographs were taken, and cell viability was analysed by MTT assay. Results are expressed as percentage of control and are means±SEM (n=3). Significance of differences from control is expressed as ***p<0.001 (ANOVA).

FIG. 3. Comparison of the effect of TAT-Cx43₂₆₆₋₂₈₃ versus TAT-Cx43₂₆₆₋₂₈₃ on the survival and proliferation of human glioblastoma stem cells. G166 human glioblastoma stem cells were treated in the absence (control) or presence of TAT, TAT-Cx43₂₆₆₋₂₈₃ or TAT-Cx43₂₆₈₋₂₈₃, all at a concentration of 50 μM. At 72h after the start of treatment, cell viability was analysed by MTT assay. Results are expressed as percentage of control and are means±SEM (n=3). Significance of differences from the control condition is expressed as ***p<0.001 and ###p<0.001 from the TAT condition (ANOVA).

FIG. 4. Effect of phenylalanine 268 of the Cx43 sequence 266-283 on the survival and proliferation of human glioblastoma stem cells. G166 human glioblastoma stem cells were treated in the absence (control) or presence of TAT-Cx43₂₆₆₋₂₈₃ or the same peptide in which phenylalanine 268 was replaced by alanine (TAT-Cx43₂₆₆₋₂₈₃ F/A), both at a concentration of 50 μM. At 48h after the start of treatment, photomicrographs were taken and cell viability was analysed by MTT assay. Results are normalised against the control (value 1) and are means±SEM (n=3). Significance of differences from the control situation is expressed as ***p<0.001 and ###p<0.001 TAT-Cx43₂₆₆₋₂₈₃ vs TAT-Cx43₂₆₆-283 F/A (ANOVA).

FIG. 5. Representative images of the effect of Cx43-based penetrant peptides, in which amino acids of the consensus SH3-binding region have been reduced, in human glioblastoma stem cells. G166 human glioblastoma stem cells were treated in the absence (control) or presence of TAT, TAT-Cx43₂₇₄-291, TAT-Cx43₂₆₆₋₂₈₃, TAT-Cx43₂₆₆₋₂₈₁, TAT-Cx43₂₆₆₋₂₇₉, TAT-Cx43₂₆₆₋₂₇₇ and TAT-Cx43₂₆₆₋₂₇₅, all at a concentration of 50 μM. Representative phase-contrast images of the cells 48h after the start of treatment are shown. Note that peptides containing Cx43 sequences 266-283, 266-281, 266-279, 266-277 and 266-275 reduce the survival and proliferation of G166 human glioblastoma stem cells, while TAT peptides and TAT-Cx43₂₇₄₋₂₉₁ do not affect the viability and proliferation of these cells when compared to the control.

FIG. 6. Effect of Cx43-based penetrant peptides, in which amino acids of the consensus SH3-binding region have been reduced, on the survival and proliferation of human glioblastoma stem cells. G166 human glioblastoma stem cells were treated in the absence (control) or presence of TAT, TAT-Cx43₂₇₄-291, TAT-Cx43₂₆₆₋₂₈₃, TAT-Cx43₂₆₆₋₂₈₁, TAT-Cx43₂₆₆₋₂₇₉, TAT-Cx43₂₆₆₋₂₇₇ and TAT-Cx43₂₆₆₋₂₇₅, all at a concentration of 50 μM. At 0, 24, 48 and 72h after the start of treatment, cell viability was analysed by MTT assay. Results are normalised to the values obtained at time 0 (dotted line value 1) and are means±SEM (n=3). Significance of differences with respect to the control condition is expressed as ***p<0.001; **p<0.01 and with respect to the TAT-Cx43 condition₂₆₆₋₂₈₃ as ##p<0.01 and #p<0.05 (ANOVA).

FIG. 7. Representative images of the effect of Cx43-based penetrating peptides on the migration of human glioblastoma stem cells. G166 human glioblastoma stem cells were treated in the absence (control) or presence of TAT-Cx43₂₆₆₋₂₈₃ or TAT-Cx43₂₆₆₋₂₇₅, both at a concentration of 50 μM. Phase-contrast videos of the cells were made using a live cell microscope. Shown are 3 representative frames taken every 100 minutes of the cells in the control condition, TAT-Cx43₂₆₆₋₂₈₃ and TAT-Cx43₂₆₆₋₂₇₅. 3 cells in each condition have been selected and numbered. Note that in the control condition the cell location changes are greater than in cells treated with the peptides TAT-Cx43₂₆₆₋₂₈₃ or TAT-Cx43₂₆₆₋₂₇₅, indicating that these peptides affect glioma stem cell migration.

FIG. 8. TAT-Cx43₂₆₆₋₂₈₃ and TAT-Cx43₂₆₆₋₂₇₅ bind to albumin via a cysteine dependent mechanism. TAT, TAT-Cx43₂₇₄₋₂₉₁, TAT-Cx43₂₆₆₋₂₈₃, TAT-Cx43₂₆₆₋₂₈₃F/A, TAT-Cx43₂₆₆₋₂₈₃C/A and TAT-Cx43₂₆₆₋₂₇₅ were fused to biotin to allow their analysis by Western blot. Solutions of each peptide plus albumin in PBS were analyzed in native (N) or denaturing and reducing conditions (DR) to identify their interaction with albumin. A) Ponceau staining shows the electrophoretic profile of albumin under native (N) or denaturing and reducing conditions (DR). Note that the sensitivity of the Ponceau staining was not enough to detect the peptide at about 10 kDa, therefore only albumin bands are shown. B) To visualize the biotinylated peptides the membrane was incubated with streptavidin, which allowed the identification of albumin-bound peptides under native conditions with the same profile showed by albumin in the Ponceau staining (from 50 to 150 kDa) and the unbound peptides at about ≤10 kDa under native or denaturing and reducing conditions. Note that TAT-Cx43₂₆₆₋₂₈₃ and TAT-Cx43₂₆₆₋₂₇₅ appear bound to albumin under native conditions while denaturation and reduction promoted the release of these peptides. Substitution of phenylalanine by alanine (TAT-Cx43₂₆₆₋₂₈₃F/A) did not affect albumin-peptide interaction. However, cysteine by alanine substitution (TAT-Cx43₂₆₆₋₂₈₃C/A) disrupted albumin-peptide interaction, indicating that cysteine at position 271 mediates this interaction.

FIG. 9. Intracellular interactions of biotinylated TAT-Cx43₂₆₆₋₂₈₃, TAT-Cx43₂₆₆₋₂₈₃C/A and TAT-Cx43₂₆₆₋₂₇₅ in human glioblastoma stem cells. Human E22 glioblastoma stem cells were incubated with 50 μM biotinylated TAT-Cx43₂₆₆₋₂₈₃, TAT-Cx43₂₆₆₋₂₈₃C/A and TAT-Cx43₂₆₆₋₂₇₅. After 30 min the cells were lysed and biotinylated peptides attached to their intracellular partners were pulled down with NeutrAvidin beads. The eluted proteins were loaded and analyzed by Western blot to study the levels of c-Src, Y416-Src, and Csk. Note that c-Src, Y416-Src, and Csk interact preferentially with TAT-Cx43₂₆₆₋₂₈₃ and TAT-Cx43₂₆₆₋₂₇₅ but they did not show significant interactions with TAT-Cx43₂₆₆₋₂₈₃C/A.

### EXAMPLES

In the following pages, the invention will be illustrated by experiments carried out by the inventors, which demonstrate the effectiveness of the invention.

### 1. Materials and Methods

### 1.1. Human Glioblastoma Stem Cell Cultures

The human glioblastoma stem cell line G166 was obtained from Biorep, deposited and characterised in Pollard et al., Cell Stem Cell. 4(6):568-80 (2009). The human glioblastoma stem cell line E22 was also obtained from Pollard lab (U. Edinburgh). These cells were obtained from glioblastoma patients after surgery and exhibit the characteristics of human glioblastoma stem cells, including the capacity for self-renewal, differentiation into distinct neural lineages and tumourigenicity.

In all these cases, cells were cultured in neurobasal culture medium supplemented with 2% B27, 1% N2, 20 ng/mL EGF and 20 ng/mL bFGF, in laminin-coated culture plates, to generate adherent cell cultures. Thanks to their self-renewal capacity, when these cells are expanded in adherent cultures under these culture conditions, they maintain the above-mentioned properties, i.e. the characteristic glioblastoma stem cell phenotype and genotype, including their tumourigenicity, for more than 20 passages.

1.2. Treatment with Cx43-Based Penetrating Peptides.

Different peptides based on the 266-283 sequence of Cx43 fused to TAT at its N-terminal end were used, all at a concentration of 50 μM, in culture medium and their effect was analysed at different treatment times. In parallel, these cells were analysed in the absence of treatment (control), in the presence of the antitumour peptide TAT-Cx43₂₆₆₋₂₈₃ (YGRKKRRQRRRAYFNGCSSPTAPLSPMSP; corresponding to the peptide described in patent WO2014191608 A1 linked to the cell internalisation sequence TAT; SEQ ID NO: 35) and in the presence of the peptides without antitumour activity, TAT (YGRKKRRQRRR; SEQ ID NO: 7) and TAT-Cx43₂₇₄₋₂₉₁ (YGRKKRRQRRR PTAPLSPMSPPGYKLVTG; SEQ ID NO: 26). For pull-down assays and visualization the peptides were fused to biotin via a C-terminal lysine.

The sequences studied were:

Cx43₂₆₆₋₂₈₃ C/A:
(SEQ ID NO: 27)
AYFNGASSPTAPLSPMSP
Cx43₂₇₁₋₂₈₇:
(SEQ ID NO: 28)
CSSPTAPLSPMSPPGYK
Cx43₂₆₈₋₂₈₃:
(SEQ ID NO: 29)
FNGCSSPTAPLSPMSP
Cx43₂₆₆₋₂₈₃ F/A:
(SEQ ID NO: 30)
AYANGCSSPTAPLSPMSP
Cx43₂₆₆₋₂₈₁:
(SEQ ID NO: 6)
AYFNGCSSPTAPLSPM
Cx43₂₆₆₋₂₇₉:
(SEQ ID NO: 5)
AYFNGCSSPTAPLS
Cx43₂₆₆₋₂₇₇:
(SEQ ID NO: 4)
AYFNGCSSPTAP
Cx43₂₆₆₋₂₇₅:
(SEQ ID NO: 1)
AYFNGCSSPT

In all cases, the study was performed with these sequences linked to the penetrant peptide YGRKKRRQRRR (SEQ ID NO: 7):

TAT-Cx43₂₆₆₋₂₈₃ C/A:
(SEQ ID NO: 31)
YGRKKRRQRRRAYFNGASSPTAPLSPMSP
TAT-Cx43₂₇₁₋₂₈₇:
(SEQ ID NO: 32)
YGRKKRRQRRRCSSPTAPLSPMSPPGYK
TAT-Cx43₂₆₈₋₂₈₃:
(SEQ ID NO: 33)
YGRKKRRQRRRFNGCSSPTAPLSPMSP
TAT-Cx43₂₆₆₋₂₈₃ F/A:
(SEQ ID NO: 34)
YGRKKRRQRRRAYANGCSSPTAPLSPMSP
TAT-Cx43₂₆₆₋₂₈₁:
(SEQ ID NO: 11)
YGRKKRRQRRRAYFNGCSSPTAPLSPM
TAT-Cx43₂₆₆₋₂₇₉:
(SEQ ID NO: 10)
YGRKKRRQRRRAYFNGCSSPTAPLS
TAT-Cx43₂₆₆₋₂₇₇:
(SEQ ID NO: 9)
YGRKKRRQRRRAYFNGCSSPTAP
TAT-Cx43₂₆₆₋₂₇₅:
(SEQ ID NO: 8)
YGRKKRRQRRRAYFNGCSSPT

### 1.3. MTT Colour Viability Test

For this study, cells were seeded at low density and the number of viable cells was determined at different days of treatment. For this purpose, the medium was removed and the cells were incubated with 300 μL of PBS in the presence of MTT (0.5 mg/mL) for 75 minutes in the dark at 37° C. in a CO₂ incubator. The medium was then removed, and 500 μL of DMSO was added. The cells were shaken in the dark for 10 minutes. Finally, absorbance was determined at 570 nm.

### 1.4. Western Blotting

Western blotting was performed as described previously (Jaraiz-Rodriguez, M. et al., Neuro-Oncology, 22, 493-504 (2020)). Briefly, 2.5 μM biotinylated peptides plus 0.1% (w/v) albumin in PBS were analysed by Western blot under native (N) or denaturing and reducing (DR) conditions. For native conditions, samples were analysed without further treatment. For denaturing and reducing conditions samples were treated with 0.1 M β-mercaptoethanol and 4% SDS and heated at 99° C. for 5 min. Samples were subjected to electrophoresis on NuPAGE Novex Bis-Tris 4-12% Midi gels (Life Technologies) at room temperature and constant voltage. Proteins were transferred to a nitrocellulose membrane (iBlot Gel Transfer Stacks Nitrocellulose) using an iBlot dry blotting system (Life Technologies). Membranes were incubated with Ponceau S staining solution for protein staining in red. After washing the membranes were blocked with 5% non-fat dry milk in PBS and then incubated with HRP-Streptavidin (1:5000; Dako P0397) for 30 minutes at room temperature. After extensive washing, the membranes were developed with a chemiluminescent substrate (Western Blotting Luminol Reagent; Santa Cruz Biotechnology) in a MicroChemi imaging system (Bioimaging Systems).

### 1.5. Pull-Down Experiments

Pulldown experiments were performed as described in González-Sánchez A. et al., Oncotarget, 7(31):49819-49833 (2016); Jaraiz-Rodriguez M. et al., J Vis Exp. (130):56457 (2017)). Confluent cells grown in 25 cm² flasks were incubated with 50 μM biotinylated peptides for 30 min. Proteins were then collected in 0.5 ml of lysis buffer (20 mM Tris-HCl (pH 8.0), 137 mM NaCl, 1% IGEPAL, 1 mM PMSF, protease cocktail (1:100; Cocktail Ill, Calbiochem), 1 mM NaF and 0.1 mM Na₃VO₄). Lysates were centrifuged at 11000×g for 10 minutes at 4° C., and the supernatants were recovered. A 50-μl aliquot of each lysate was used to analyze the protein content, and the remaining lysate was incubated with NeutrAvidin-Agarose (Thermo Scientific, Rockford, IL, USA; Ref. 29200) for 12 h at 4° C. with gentle shaking. The avidin beads bound with the peptides were collected by centrifugation (3000×g for 1 minutes at 4° C.). The beads were then washed five times with lysis buffer, and the bound proteins were eluted and analyzed by Western blotting under denaturing and reducing contidions. The membranes were blocked with 5% non-fat dry milk in PBS-T and then incubated overnight at 4° C. with the primary antibodies against rabbit CSK (1:250; Cell Signaling Technology, Ref. 4980), rabbit Y416 Src (1:250, Cell Signaling Technology, Ref. 2101) and mouse total c-Src (1:500, Cell Signaling Technology, Ref. 2110). To detect biotinylated peptides, the membranes were incubated with HRP-conjugated streptavidin in PBS (1:5000; Dako Ref.P0397) for 30 minutes and then developed with a chemiluminescent substrate.

### 2. Results

The following results show the existence of critical amino acids in the 266-274 region for the anti-tumour effect of Cx43.

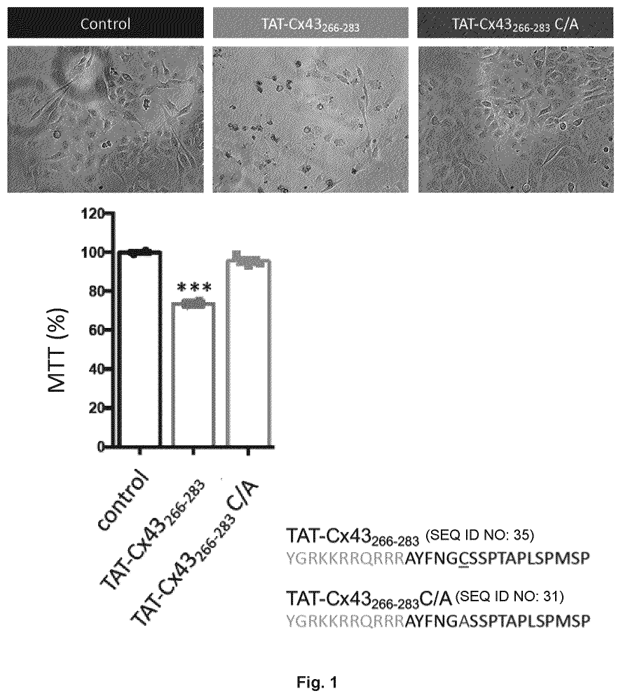

In particular, the cysteine (C) at position 271 is essential for this effect. Thus, replacing this cysteine with an alanine (A) causes the loss of the antitumour effect (FIG. 1). FIG. 1 shows that the morphology and viability of glioblastoma stem cells treated with TAT-Cx43₂₆₆₋₂₈₃ C/A (TAT-AYFNGASSPTAPLSPMSP (SEQ ID NO: 31)) is very similar to that of untreated (control) cells, whereas TAT-Cx43₂₆₆₋₂₈₃ significantly reduces the viability of these tumour cells. These results demonstrate the relevance of cysteine 271 for the antitumour effect of the 266-283 sequence in Cx43.

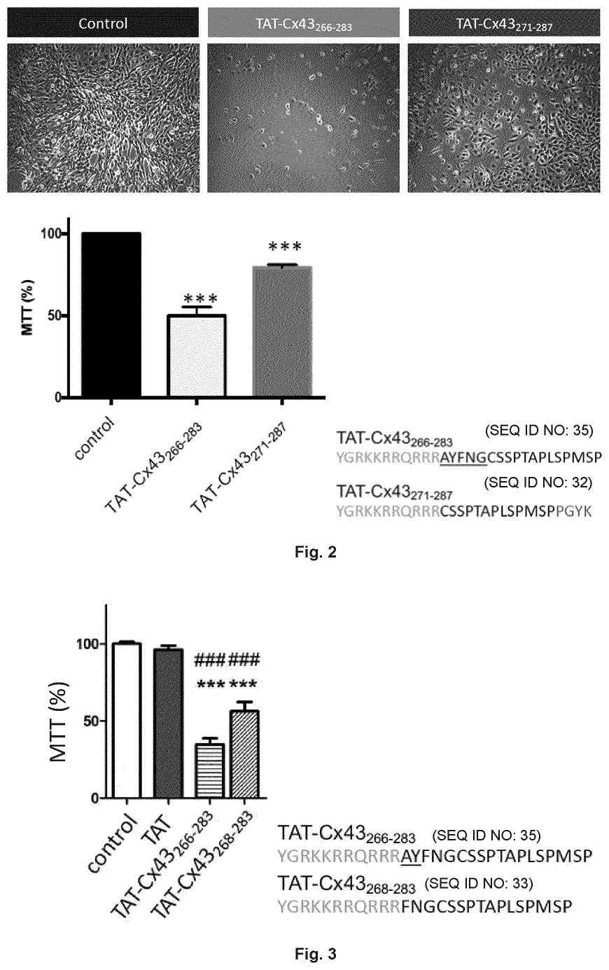

In order to determine whether the presence of cysteine alone is sufficient for the activity, we analyzed the effect of the 271-287 sequence containing cysteine 271, whose effect in interfering with the closure by acidification of the gap junctions formed by Cx43 has been previously described (Calero G., Circ Res. Circ Res., 82(9): 929-35 (1998)). FIG. 2 shows that treatment with the TAT-Cx43₂₇₁₋₂₈₇ peptide (SEQ ID NO: 32) slightly reduces glioblastoma stem cell proliferation, but its effect is much lower than that of TAT-Cx43₂₆₆₋₂₈₃. These results demonstrate that cysteine 271 is necessary, but not sufficient for the anti-tumour effect.

Indeed, deletion of amino acids 266 and 267 affects the antitumour effect (FIG. 3). The TAT-Cx43 peptide₂₆₈₋₂₈₃ (TAT-FNGCSSPTAPLSPMSP (SEQ ID NO: 33)) has less ability to reduce proliferation than TAT-Cx43₂₆₆₋₂₈₃ (TAT-AYFNGCSSPTAPLSPMSP), indicating the importance of amino acids at positions 266 and 267 for the antitumour effect of this peptide.

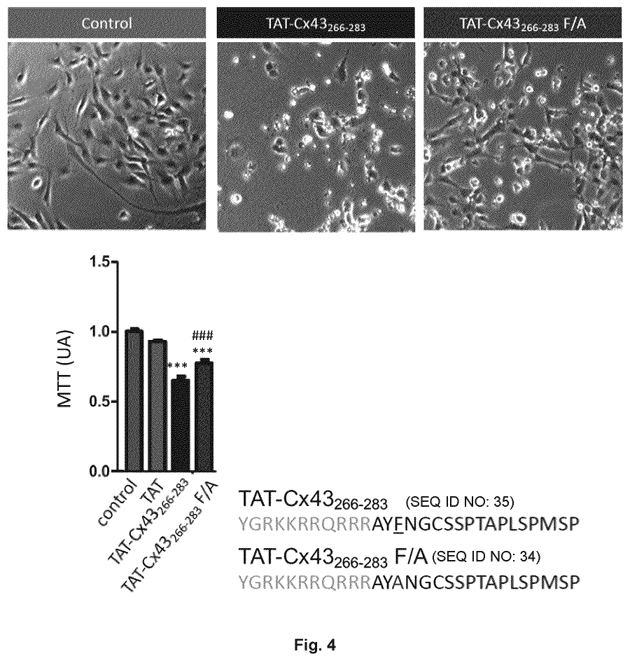

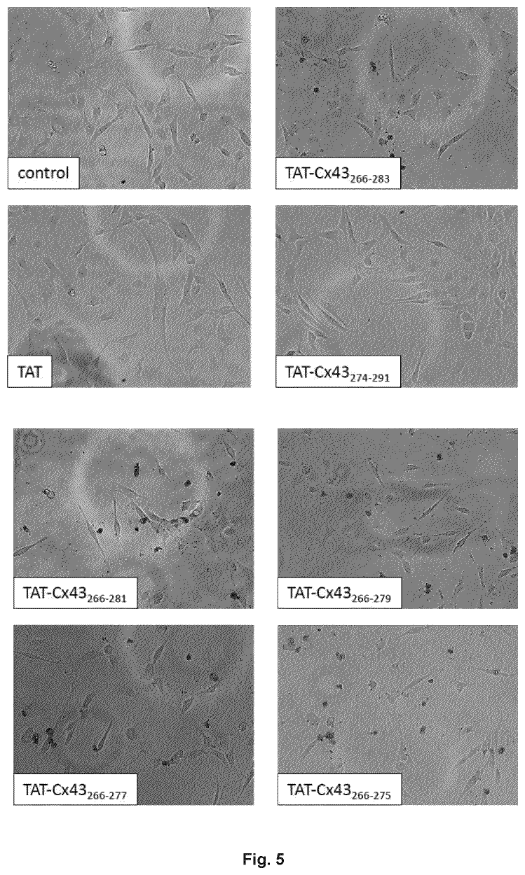

The substitution of phenylalanine (F) with an alanine (A) at position 268 (TAT-Cx43₂₆₆₋₂₈₃ F/A: TAT-AYANGCSSPTAPLSPMSP (SEQ ID NO: 34)) also reduces the antitumour effect. FIG. 4 shows how the penetrant peptide TAT-Cx43₂₆₆₋₂₈₃ F/A affects tumour cell morphology and viability, but to a much lesser extent than the TAT-Cx43 peptide₂₆₆₋₂₈₃.

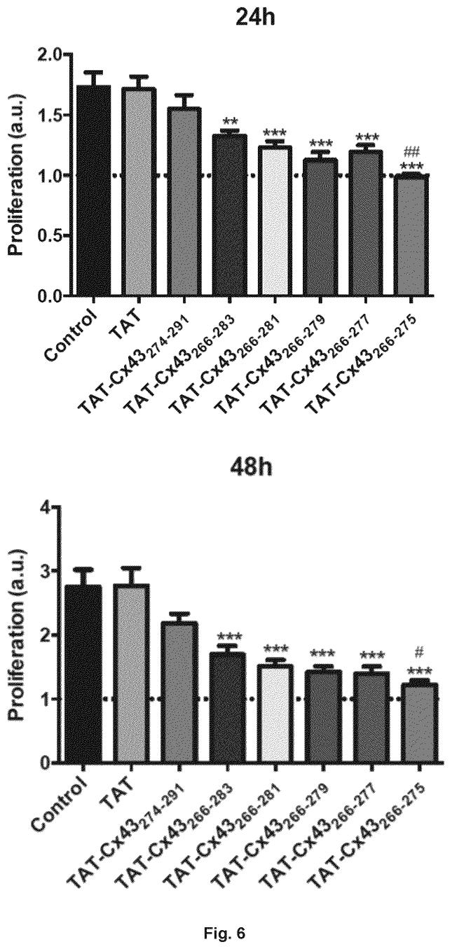

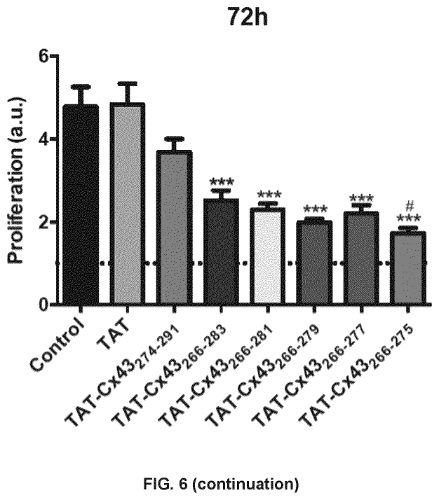

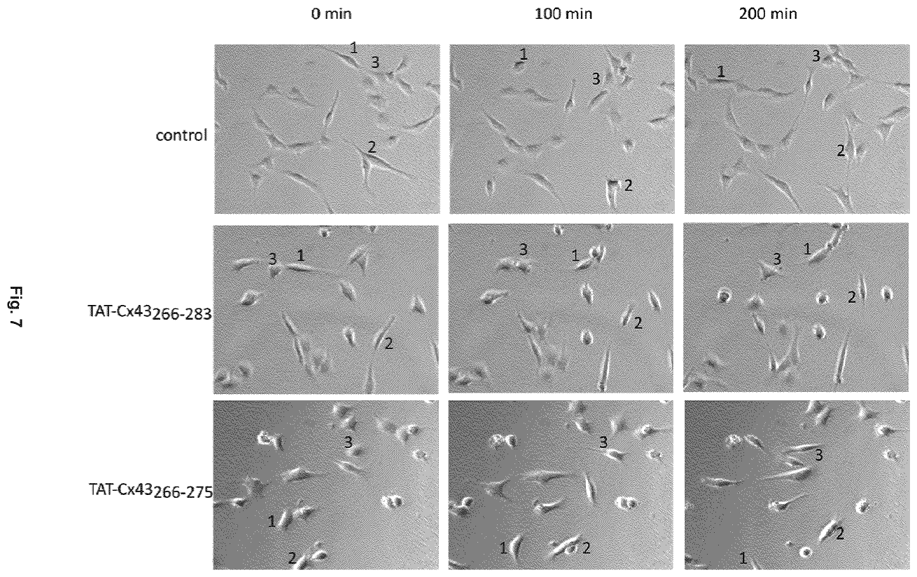

On the other hand, the relevance of amino acids in the proline-rich consensus region for the antitumour effect was analysed. For this purpose, different peptides reducing the sequence from the C-terminal end were used: TAT-Cx43₂₆₆₋₂₈₁ (TAT-AYFNGCSSPTAPLSPM (SEQ ID NO: 11)), TAT-Cx43₂₆₆₋₂₇₉ (TAT-AYFNGCSSPTAPLS (SEQ ID NO: 10)), TAT-Cx43₂₆₆₋₂₇₇ (TAT-AYFNGCSSPTAP (SEQ ID NO: 9)) and TAT-Cx43₂₆₆₋₂₇₅ (TAT-AYFNGCSSPT (SEQ ID NO: 8)). As shown in FIG. 5, the results show that the penetrant peptides: TAT-Cx43₂₆₆₋₂₈₁, TAT-Cx43₂₆₆₋₂₇₉, TAT-Cx43₂₆₆₋₂₇₇ and TAT-Cx43₂₆₆₋₂₇₅ reduce proliferation and survival of human glioblastoma stem cells, similar to what was observed with the TAT-Cx43₂₆₆₋₂₈₃ peptide. In fact, the TAT-Cx43₂₆₆₋₂₇₅ peptide has an anti-tumour effect significantly higher than the rest of the peptides (FIG. 6). By contrast, the cell penetrant peptides TAT-Cx43₂₇₄₋₂₉₁ (SEQ ID NO: 26) or TAT do not produce significant effects on G166 glioblastoma stem cells (FIGS. 5 and 6).

Using these same peptides, but fused to biotin, we confirmed that TAT-Cx43₂₆₆₋₂₈₁, TAT-Cx43₂₆₆₋₂₇₉, TAT-Cx43₂₆₆₋₂₇₇ and TAT-Cx43₂₆₆₋₂₇₅ were able to be internalised in human glioblastoma stem cells in a similar way to TAT-Cx43₂₆₆₋₂₈₃.

Furthermore, images taken at different times using a live cell microscope showed that the TAT-Cx43₂₆₆₋₂₇₅ peptide reduces the migration of human glioblastoma stem cells, similarly to what was observed with TAT-Cx43₂₆₆₋₂₈₃ (FIG. 7). Migration is the cause of these tumour cells invading the surrounding parenchyma, escaping surgery and causing the recurrence of these tumours, which is the main cause of mortality.

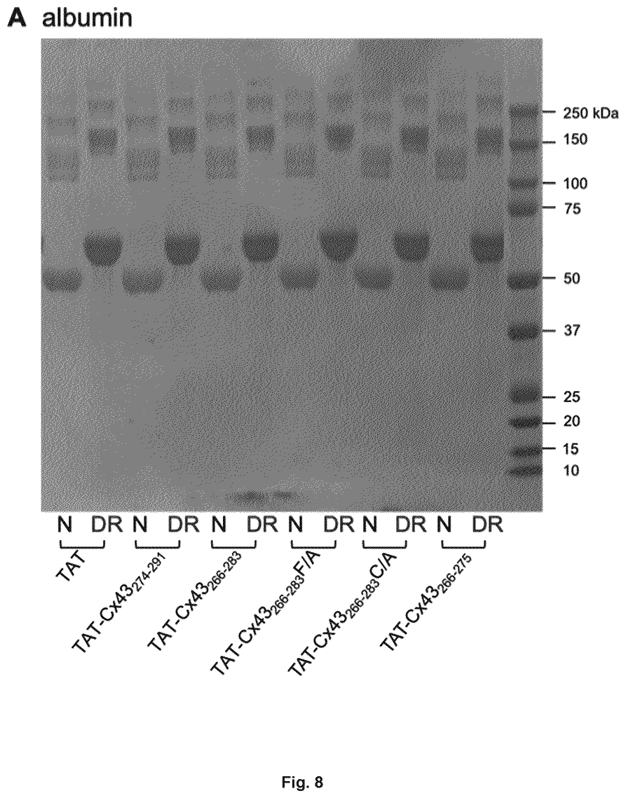

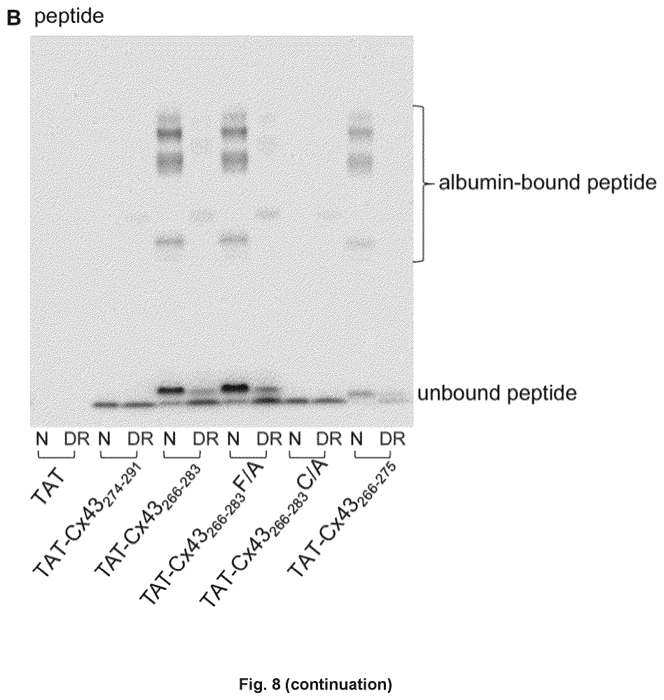

One of the main advantages of peptide-based drugs, such as those described in this invention, is their low toxicity and high specificity (Otvos, L. and Wade, J. 2014. Frontiers in Chemistry, volume 2; doi: 10.3389/fchem.2014.00062). However, peptide-based drugs have a rapid renal clearance and their degradation by endogenous proteases result in poor pharmacokinetic profile, which limit their therapeutical use. Importantly, our results show that TAT-Cx43₂₆₆₋₂₈₃ and TAT-Cx43₂₆₆₋₂₇₅ bind to albumin (FIG. 8), the most abundant protein in plasma, which may strongly improve their pharmacokinetic properties. Indeed, albumin-binding protects peptide-based drugs from endogenous proteases and decreases their renal clearance, improving the pharmacokinetic profile of these peptides (Liu and Xiaoyuan Chen. 2016. Chem Soc Rev. 2016 Mar. 7; 45(5): 1432-1456. doi:10.1039/c5cs00158g.).

As shown in FIG. 8, solutions of albumin plus biotinylated peptides (TAT, TAT-Cx43₂₇₄₋₂₉₁, TAT-Cx43₂₆₆₋₂₈₃, TAT-Cx43₂₆₆₋₂₈₃F/A, TAT-Cx43₂₆₆₋₂₈₃C/A or TAT-Cx43₂₆₆₋₂₇₅) were analyzed by Western blot under native (N) or denaturing and reducing (DR) conditions to determine their ability to bind to albumin. Under native conditions, TAT-Cx43₂₆₆₋₂₈₃ and TAT-Cx43₂₆₆₋₂₇₅ show the same profile than albumin in the Ponceau staining (different bands from 50 to 150 kDa), indicating that these peptides bind to albumin under native conditions. Interestingly, the substitution of phenylalanine by alanine (TAT-Cx43₂₆₆₋₂₈₃F/A) did not affect the interaction with albumin. However, the substitution of cysteine by alanine (TAT-Cx43₂₆₆₋₂₈₃C/A) disrupted albumin-peptide interaction as judged by the absence of peptide signal from 50 to 150 kDa under native conditions. These results suggest that cysteine at position 271 mediates the interaction of these peptides with albumin, presumably through a disulfide bond with the free cysteine residue in albumin (Nakashima, F., Shibata, T., Kamiya, K. et al. 2018. Sci Rep 8, 932. https://doi.org/10.1038/s41598-018-19610-9). To confirm these results the samples were denatured by heating with SDS under reducing conditions (DR). Western blot showed the presence of the peptides at about 10 kDa, indicating the release of their binding to albumin under denatured and reduced conditions (DR). Together, these results show that TAT-Cx43₂₆₆₋₂₈₃ and TAT-Cx43₂₆₆₋₂₇₅ peptides bind to albumin through a disulfide bond among the cysteine at position 271 and the free cysteine residue in albumin.

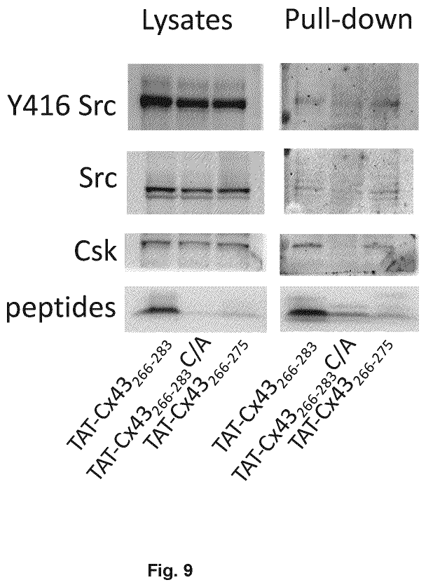

Previous results showed that the sequence 266-283 from Cx43 recruits c-Src, its active form, Y416-Src, and its endogenous inhibitor, Csk, which promotes the inhibition of the oncogenic activity of c-Src (Gonzalez-Sanchez A. et al., Oncotarget, 7(31):49819-49833 (2016); Jaraiz-Rodriguez M. et al., J Vis Exp. (130):56457 (2017). In the present study, the inventors demonstrated that the region 266-275 is sufficient to recruit c-Src, its active form, Y416-Src, and its endogenous inhibitor, Csk. FIG. 9 shows the results of pulldown assays after the incubation of biotinylated TAT-Cx43₂₆₆₋₂₈₃, TAT-Cx43₂₆₆₋₂₈₃C/A and TAT-Cx43₂₆₆₋₂₇₅ in human glioblastoma stem cells. As described before, the region 266-283 recruits c-Src, Y416-Src and Csk. Importantly, these intracellular interactions were significantly reduced when the cysteine at position 271 was substituted by alanine (TAT-Cx43₂₆₆₋₂₈₃C/A). Furthermore, the region 266-275 was sufficient to recruit c-Src, Y416-Src and its endogenous inhibitor, Csk.

### 3. Conclusions

1. The cysteine at position 271 in the 266-283 sequence of Cx43 is required for the antitumour effect of this sequence, as its substitution by an alanine results in the loss of this effect, as can be seen in FIG. 1 wherein it is shown that the effect reached by the peptide comprising the substitution C271A is not significatively different with respect to the control.

2. The cysteine at position 271 in the sequence 266-283 of Cx43 is not sufficient for the anti-tumour effect, as the sequence 271-287 has a much lower effect than that presented by the sequence 266-283.

3. The elimination of amino acids at positions 266 and 267 in the sequence 266-283 of Cx43 reduces the anti-tumour effect.

4. The substitution of phenylalanine at position 268 in the sequence 266-283 of Cx43 by alanine reduces the anti-tumour effect of this sequence.

5. Amino acids from positions 276 to 283 of the sequence 266-283 of Cx43 are not necessary for the antitumour effect of this sequence, as their elimination does not affect this effect.

6. The antitumour effect of the TAT-Cx43₂₆₆₋₂₇₅ peptide is significantly higher than that observed with TAT-Cx43₂₆₆₋₂₈₃.

7. TAT-Cx43₂₆₆₋₂₈₃ and TAT-Cx43₂₆₆₋₂₇₅ bind to albumin, which protects the peptides from proteases and prevents a rapid renal clearance. Cysteine at position 271 is required for peptide-albumin binding, presumably through a disulfide bond.

8. The region 266-275 is sufficient to recruit c-Src, its active form Y416-Src, and the Src inhibitor, Csk. Cysteine at position 271 is required for these interactions.

Thus, these results demonstrate that the antitumour effect resides in the 266-275 sequence of Cx43 and that cysteine 271 is required for this function.

1. A peptide comprising the amino acid sequence having a sequence identity of at least 70, 75, 80, 85, 90, 91, 92, 93, 94, 95, 96, 97, 98 or 99% with SEQ ID NO: 1, with the proviso that said peptide has neither the amino acid sequence SEQ ID NO: 2 nor SEQ ID NO: 3, and that the cysteine at position 6 of SEQ ID NO: 1 remains unchanged.

2. Peptide according to claim 1, comprising the amino acid sequence having a sequence identity of 100% with SEQ ID NO: 1.

3. Peptide according to claim 1 or 2, comprising the amino acid sequence SEQ ID NO: 4.

4. Peptide according to any one of claims 1 to 3, comprising the amino acid sequence SEQ ID NO: 5.

5. Peptide according to any one of claims 1 to 4, comprising the amino acid sequence SEQ ID NO: 6.

6. Peptide according to any one of claims 1 to 5, wherein the peptide has a length of 10 to 100 amino acids.

7. Peptide according to any one of claims 1 to 6, wherein the peptide is covalently linked to a cell internalizing amino acid sequence.

8. Peptide according to claim 7, wherein the cell internalisation amino acid sequence is attached to the amino terminal end of the peptide.

9. Peptide according to claim 7 or 8, wherein the cell internalization sequence comprises the amino acid sequence SEQ ID NO: 7.

10. Peptide according to any one of claims 1 to 9, wherein the peptide comprises the amino acid sequence SEQ ID NO: 8, SEQ ID NO: 9, SEQ ID NO: 10, or SEQ ID NO: 11.

11. Peptide according to any one of claims 1 to 10, wherein the peptide is covalently bound to biotin.

12. A polynucleotide encoding a peptide according to any one of claims 1 to 11.

13. A gene construct comprising a polynucleotide according to claim 12.

14. A vector comprising a polynucleotide according to claim 12, or a gene construct according to claim 13.

15. Vector according to claim 14, wherein the vector is a viral vector, preferably a viral vector of a retrovirus, a lentivirus or an adenovirus.

16. A cell comprising a polynucleotide according to claim 12, a gene construct according to claim 13 or a vector according to claim 14 or 15.

17. A composition comprising the peptide according to any one of claims 1 to 11, a polynucleotide according to claim 12, a gene construct according to claim 13, a vector according to claim 14 or 15, or a cell according to claim 16.

18. Composition according to claim 17, further comprising a vehicle and/or a chemotherapeutic agent.

19. Composition according to claim 17 or 18, wherein the composition is formulated for oral, parenteral, nasal, sublingual or intratumoral administration.

20. Composition according to any one of claims 17 to 19, wherein the composition is a pharmaceutical composition.

21. Peptide according to any one of claims 1 to 11, polynucleotide according to claim 12, gene construct according to claim 13, vector according to claim 14 or 15, cell according to claim 16, or composition according to any one of claims 17 to 20, for use as a medicament.

22. Peptide according to any one of claims 1 to 11, polynucleotide according to claim 12, gene construct according to claim 13, vector according to claim 14 or 15, cell according to claim 16, or composition according to any one of claims 17 to 20, for use in the treatment of benign or malignant tumours.

23. A peptide, polynucleotide, gene construct, vector, cell or composition for use according to claim 22, wherein the malignancy is cancer.

24. A peptide, polynucleotide, gene construct, vector, cell or composition for use according to claim 22 or 23, wherein the tumour is a brain tumour.

25. A peptide, polynucleotide, gene construct, vector, cell or composition for use according to claim 22 to 24, wherein the tumour is a glioma.

26. A peptide, polynucleotide, gene construct, vector, cell or composition for use according to claim 25, wherein the glioma is a glioma selected from the group consisting of astrocytoma, oligodendroglioma, glioblastoma and ependymoma.

27. A peptide, polynucleotide, gene construct, vector, cell or composition for use according to any one of claims 22 to 26, wherein the tumour comprises stem cells.

28. A peptide, polynucleotide, gene construct, vector, cell or composition for use according to 27, wherein the stem cells comprise tumour stem cells.

29. Peptide according to any one of claims 1 to 11, polynucleotide according to claim 12, gene construct according to claim 13, vector according to claim 14 or 15, cell according to claim 16, or composition according to any one of claims 17 to 20, for use in preventing proliferation, migration and/or metastasis of tumour cells.

30. A peptide, polynucleotide, gene construct, vector, cell or composition for use according to claim 29, wherein the tumour cells are stem cells.

31. Peptide, polynucleotide, gene construct, vector, cell or composition for use according to claim 30, wherein the stem cells are glioma stem cells, preferably, the glioma stem cells are astrocytoma stem cells, oligodendroglioma stem cells, glioblastoma stem cells or ependymoma stem cells.

32. Use of a peptide according to any one of claims 1 to 11, a polynucleotide according to claim 12, a gene construct according to claim 13, a vector according to claim 14 or 15, a cell according to claim 16, or a composition according to any one of claims 17 to 20, to inhibit in vitro cell proliferation and/or to reverse the phenotype of tumour stem cells.

33. Use according to claim 32, wherein the tumour stem cells are glioma stem cells, preferably, the glioma stem cells are astrocytoma stem cells, oligodendroglioma stem cells, glioblastoma stem cells or ependymoma stem cells.

34. Use of a peptide according to any one of claims 1 to 11, a polynucleotide according to claim 12, a gene construct according to claim 13, a vector according to claim 14 or 15, a cell according to claim 16, or a composition according to any one of claims 17 to 20, for the in vitro identification of tumourigenicity regulatory or modulating compounds.

35. Kit comprising a peptide according to any one of claims 1 to 11, a polynucleotide according to claim 12, a gene construct according to claim 13, a vector according to claim 14 or 15, a cell according to claim 16, or a composition according to any one of claims 17 to 20.

36. Use of the kit according to claim 35, for in vitro determination of the effect of the peptide according to any one of claims 1 to 11, on the tumorigenicity of a cell line, or for in vitro inhibition of cell proliferation and/or reversal of the phenotype of tumour stem cells.

37. Use according to claim 36, wherein inhibiting cell proliferation comprises inhibiting cell proliferation of tumour stem cells, preferably, of glioma stem cells, more preferably, of astrocytoma stem cells, oligodendroglioma stem cells, glioblastoma stem cells or ependymoma stem cells.


SEQUENCE LISTING (35 sequences)

SEQ ID NO: 1 - protein, 10 aa, synthetic construct
  REGION 1..10: note = Peptide
AYFNGCSSPT

SEQ ID NO: 2 - protein, 128 aa, synthetic construct
  REGION 1..128: note = Aminoacid sequence from the carboxyl-terminal end of Connexin43
SPSKDCGSPKYAYFNGCSSPTAPLSPMSPPGYKLVTGDRNNSSCRNYNKQASEQNWANYSAEQNRMGQAGSTISNSHAQPFDFPDDNQNAKKVAAGHELQPLAIVDQRPSSRASSRASSRPRPDDLEI

SEQ ID NO: 3 - protein, 18 aa, synthetic construct
  REGION 1..18: note = Peptide
AYFNGCSSPTAPLSPMSP

SEQ ID NO: 4 - protein, 12 aa, synthetic construct
  REGION 1..12: note = Peptide
AYFNGCSSPTAP

SEQ ID NO: 5 - protein, 14 aa, synthetic construct
  REGION 1..14: note = Peptide
AYFNGCSSPTAPLS

SEQ ID NO: 6 - protein, 16 aa, synthetic construct
  REGION 1..16: note = Peptide
AYFNGCSSPTAPLSPM

SEQ ID NO: 7 - protein, 11 aa, synthetic construct
  REGION 1..11: note = Cell internalisation sequence
YGRKKRRQRRR

SEQ ID NO: 8 - protein, 21 aa, synthetic construct
  REGION 1..21: note = Peptide
YGRKKRRQRRRAYFNGCSSPT

SEQ ID NO: 9 - protein, 23 aa, synthetic construct
  REGION 1..23: note = Peptide
YGRKKRRQRRRAYFNGCSSPTAP

SEQ ID NO: 10 - protein, 25 aa, synthetic construct
  REGION 1..25: note = Peptide
YGRKKRRQRRRAYFNGCSSPTAPLS

SEQ ID NO: 11 - protein, 27 aa, synthetic construct
  REGION 1..27: note = Peptide
YGRKKRRQRRRAYFNGCSSPTAPLSPM

SEQ ID NO: 12 - protein, 16 aa, synthetic construct
  REGION 1..16: note = Cell internalisation sequence
RQIKIWFQNRRMKWKK

SEQ ID NO: 13 - protein, 12 aa, synthetic construct
  REGION 1..12: note = Cell internalisation sequence
VKKKKIKREIKI

SEQ ID NO: 14 - protein, 7 aa, synthetic construct
  REGION 1..7: note = Cell internalisation sequence
FFLIPKG

SEQ ID NO: 15 - protein, 15 aa, synthetic construct
  REGION 1..15: note = Cell internalisation sequence
DSLKSYWYLQKFSWR

SEQ ID NO: 16 - protein, 15 aa, synthetic construct
  REGION 1..15: note = Cell internalisation sequence
KLWMRWWSPTTRRYG

SEQ ID NO: 17 - protein, 15 aa, synthetic construct
  REGION 1..15: note = Cell internalisation sequence
RLWMRWYSPWTRRWG

SEQ ID NO: 18 - protein, 15 aa, synthetic construct
  REGION 1..15: note = Cell internalisation sequence
RLIMRIYAPTTRRYG

SEQ ID NO: 19 - protein, 15 aa, synthetic construct
  REGION 1..15: note = Cell internalisation sequence
RLYMRYYSPTTRRYG

SEQ ID NO: 20 - protein, 15 aa, synthetic construct
  REGION 1..15: note = Cell internalisation sequence
RLWMRWYSPRTRAYG

SEQ ID NO: 21 - protein, 15 aa, synthetic construct
  REGION 1..15: note = Cell internalisation sequence
KRPTMRFRYTWNPMK

SEQ ID NO: 22 - protein, 15 aa, synthetic construct
  REGION 1..15: note = Cell internalisation sequence
WKCRRQCFRVLHHWN

SEQ ID NO: 23 - protein, 15 aa, synthetic construct
  REGION 1..15: note = Cell internalisation sequence
WKCRRQAFRVLHHWN

SEQ ID NO: 24 - protein, 15 aa, synthetic construct
  REGION 1..15: note = Cell internalisation sequence
WKARRQAFRVLHHWN

SEQ ID NO: 25 - protein, 24 aa, synthetic construct
  REGION 1..24: note = Cell internalization sequence
YSSYSAPVSSSLSVRRSYSSSSGS

SEQ ID NO: 26 - protein, 29 aa, synthetic construct
  REGION 1..29: note = Peptide
YGRKKRRQRRRPTAPLSPMSPPGYKLVTG

SEQ ID NO: 27 - protein, 18 aa, synthetic construct
  REGION 1..18: note = Peptide
AYFNGASSPTAPLSPMSP

SEQ ID NO: 28 - protein, 17 aa, synthetic construct
  REGION 1..17: note = Peptide
CSSPTAPLSPMSPPGYK

SEQ ID NO: 29 - protein, 16 aa, synthetic construct
  REGION 1..16: note = Peptide
FNGCSSPTAPLSPMSP

SEQ ID NO: 30 - protein, 18 aa, synthetic construct
  REGION 1..18: note = Peptide
AYANGCSSPTAPLSPMSP

SEQ ID NO: 31 - protein, 29 aa, synthetic construct
  REGION 1..29: note = Peptide
YGRKKRRQRRRAYFNGASSPTAPLSPMSP

SEQ ID NO: 32 - protein, 28 aa, synthetic construct
  REGION 1..28: note = Peptide
YGRKKRRQRRRCSSPTAPLSPMSPPGYK

SEQ ID NO: 33 - protein, 27 aa, synthetic construct
  REGION 1..27: note = Peptide
YGRKKRRQRRRFNGCSSPTAPLSPMSP

SEQ ID NO: 34 - protein, 29 aa, synthetic construct
  REGION 1..29: note = Peptide
YGRKKRRQRRRAYANGCSSPTAPLSPMSP

SEQ ID NO: 35 - protein, 29 aa, synthetic construct
  REGION 1..29: note = Peptide
YGRKKRRQRRRAYFNGCSSPTAPLSPMSP